(19) United States
(12) Patent Application Publication
(10) Pub. No.: US 2007/0112033 A1
(43) Pub. Date: May 17, 2007

(54) Beta-agonists, methods for the preparation thereof and their use as pharmaceutical compositions

(76) Inventors: Thomas Trieselmann, Warthausen (DE); Rainer Walter, Biberach (DE); Matthew R. Netherton, Danbury, CT (US); Marco Santagostino, Mittelbiberach (DE); Bradford S. Hamilton, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/550,827

(22) Filed: Oct. 19, 2006

(30) Foreign Application Priority Data
Oct. 28, 2005 (DE) 102005052102

(51) Int. Cl. C07D 403/02; A61K 31/4439; A61K 31/4184

(52) U.S. Cl. 514/338; 514/394; 548/307.4; 546/273.4

(57) ABSTRACT

The present invention relates to new beta-agonists of general formula (I) 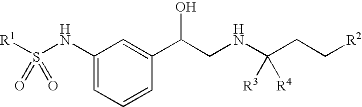
wherein the groups R¹ to R⁴ have the meanings given in ths claims and specification, the tautomers, racemates, enantiomers, diastereomers, solvates, hydrates, mixtures thereof, the prodrugs thereof and the salts thereof, particularly the physiologically acceptable salts thereof with inorganic or organic acids or bases, methods of preparing these compounds and their use as pharmaceutical compositions.

The present invention relates to new beta-agonists of general formula (I)

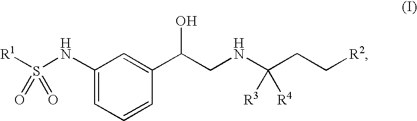

wherein the groups R¹ to R⁴ have the meanings given in the claims and specification, the tautomers, racemates, enantiomers, diastereomers, solvates, hydrates, mixtures thereof, the prodrugs thereof and the salts thereof, particularly the physiologically acceptable salts thereof with inorganic or organic acids or bases, methods for preparing these compounds and their use as pharmaceutical compositions.

### BACKGROUND OF THE INVENTION

The treatment of type II diabetes and obesity is based primarily on reducing calorie intake and increasing physical activity. These methods are rarely successful in the longer term.

It is known that beta-3 receptor agonists have a significant effect on lipolysis, thermogenesis and the serum glucose level in animal models of type II diabetes (Arch J R. beta(3)-Adrenoceptor agonists: potential, pitfalls and progress, Eur J Pharmacol. Apr. 12, 2002;440(2-3):99-107).

Compounds which are structurally similar to the compounds according to the invention and their broncholytic, spasmolytic and antiallergic activities were disclosed in DE 2833140, for example.

The aim of the present invention is to provide selective beta-3 agonists which can be used to prepare pharmaceutical compositions for the treatment of obesity and type II diabetes.

### DETAILED DESCRIPTION OF THE INVENTION

Surprisingly it has been found that compounds of general formula (I) wherein the groups R¹ to R⁴ are defined as hereinafter are effective as selective beta-3 agonists. Thus, the compounds according to the invention may be used to treat diseases connected with the stimulation of beta-3-receptors.

The present invention therefore relates to compounds of general formula (I)

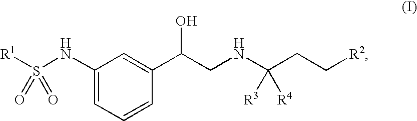

wherein

R¹ denotes a C₁₋₄-alkyl, di-(C₁₋₃-alkyl)-amino, thienyl, pyridyl or phenyl group,
wherein the phenyl group may be substituted by one to three fluorine, chlorine or bromine atoms or one to three C₁₋₃-alkyl, C₁₋₃-alkyloxy, trifluoromethoxy or difluoromethoxy groups, wherein the substituents may be identical or different

R² denotes a benzimidazolyl or 1,3-dihydrobenzimidazol-2-one group,
each of which may be substituted by one or two fluorine, chlorine or bromine atoms or one or two C₁₋₃-alkyl, hydroxy, methoxy, trifluoromethoxy, difluoromethoxy, carboxy, C₁₋₄-alkyloxy-carbonyl, ω-morpholin-4-yl-C₂₋₄-alkyloxy-carbonyl, hydrazinocarbonyl or amino groups, wherein the substituents may be identical or different or
wherein two adjacent carbon atoms may be bridged by a —CH═CH—CH═CH— group,

and R³ and R⁴, which may be identical or different, each denote a C₁₋₃-alkyl group,

while the alkyl groups contained in the above-mentioned groups may be straight-chain or branched,

and excluding the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl}-1H-benzimidazole-5-carboxylic acid and
ethyl 1-{3-[2-(3-benzenesulphonylamino-phenyl)-2-hydroxy-ethylamino]-3-methyl-butyl}-1H-benzimidazole-5-carboxylate;

optionally in the form of the tautomers, racemates, enantiomers, diastereomers, solvates and hydrates and mixtures thereof, as well as optionally the prodrugs, double prodrugs and salts thereof, particularly the physiologically acceptable salts thereof with inorganic or organic acids or bases.

Preferred compounds of general formula (I) are those wherein

R², R³ and R⁴ are as hereinbefore defined and

R¹ denotes a phenyl group, which may be substituted by a fluorine, chlorine or bromine atom or a C₁₋₃-alkyl, C₁₋₃-alkyloxy, trifluoromethoxy or difluoromethoxy group,

excluding the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl}-1H-benzimidazole-5-carboxylic acid and

ethyl 1-{3-[2-(3-benzenesulphonylamino-phenyl)-2-hydroxy-ethylamino]-3-methyl-butyl}-1H-benzimidazole-5-carboxylate;

and the tautomers, racemates, enantiomers, diastereomers, solvates, hydrates, mixtures thereof and the salts thereof,

particularly those compounds of general formula (I), wherein

R² is as hereinbefore defined,

R¹ denotes a phenyl group

and R³ and R⁴ each represent a methyl group,

excluding the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl}-1H-benzimidazole-5-carboxylic acid and

ethyl 1-{3-[2-(3-benzenesulphonylamino-phenyl)-2-hydroxy-ethylamino]-3-methyl-butyl}-1H-benzimidazole-5-carboxylate;

the tautomers, enantiomers, diastereomers, mixtures thereof and the salts thereof.

Most Particularly preferred are those compounds of general formula (I), wherein

R¹ denotes a phenyl group,

R² denotes a benzimidazol-1-yl or 1,3-dihydrobenzimidazol-2-on-1-yl group,
each of which may be substituted by one or two fluorine, chlorine or bromine atoms or a C₁₋₃-alkyl, hydroxy, methoxy, trifluoromethoxy, difluoromethoxy, carboxy, C₁₋₄-alkyloxy-carbonyl, ω-morpholin-4-yl-C₂₋₄-alkyloxy-carbonyl, hydrazinocarbonyl or amino group or
wherein two adjacent carbon atoms may be bridged by a —CH═CH—CH═CH— group,

and R³ and R⁴ each represent a methyl group,

while the alkyl groups contained in the above-mentioned groups may be straight-chain or branched,

and excluding the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl}-1H-benzimidazole-5-carboxylic acid and

ethyl 1-{3-[2-(3-benzenesulphonylamino-phenyl)-2-hydroxy-ethylamino]-3-methyl-butyl}-1H-benzimidazole-5-carboxylate;

the tautomers, enantiomers, diastereomers, mixtures thereof and the salts thereof.

Most particularly preferred are those compounds of general formula (I), wherein

R² denotes a benzimidazol-1-yl group,
which may be substituted by one or two fluorine, chlorine or bromine atoms or a C₁₋₃-alkyl, hydroxy, methoxy, trifluoromethoxy, difluoromethoxy, carboxy, C₁₋₄-alkyloxy-carbonyl, ω-morpholin-4-yl-C₂₋₄-alkyloxy-carbonyl, hydrazinocarbonyl or amino group, wherein the substituents may be identical or different or
wherein two adjacent carbon atoms may be bridged by a —CH═CH—CH═CH— group,

while the alkyl groups contained in the above-mentioned groups may be straight-chain or branched,

and excluding the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl}-1H-benzimidazole-5-carboxylic acid and

1-{3-[2-(3-benzenesulphonylamino-phenyl)-2-hydroxy-ethylamino]-3-methyl-butyl}-1H-benzimidazole-5-carboxylate ethyl;

the tautomers, enantiomers, diastereomers, mixtures thereof, the prodrugs thereof and the salts thereof;

but particularly those compounds of general formula (I), wherein

R¹ denotes a phenyl group,

R² denotes a benzimidazol-1-yl group,
each of which may be substituted by one or two fluorine, chlorine or bromine atoms or one or two C₁₋₃-alkyl, hydroxy, methoxy, carboxy, C₁₋₄-alkyloxy-carbonyl, ω-morpholin-4-yl-C₂₋₄-alkyloxy-carbonyl, or hydrazinocarbonyl groups, wherein the substituents may be identical or different or
wherein two adjacent carbon atoms may be bridged by a —CH═CH—CH═CH— group,

and R³ and R⁴ each represent a methyl group,

while the alkyl groups contained in the above-mentioned groups may be straight-chain or branched,

and excluding the compounds

1-{3-[2-(3-benzenesulphonylamino-phenyl)-2-hydroxy-ethylamino]-3-methyl-butyl}-1H-benzimidazole-5-carboxylic acid and

ethyl 1-{3-[2-(3-benzenesulphonylamino-phenyl)-2-hydroxy-ethylamino]-3-methyl-butyl}-1H-benzimidazole-5-carboxylate;

and the tautomers, racemates, enantiomers, diastereomers, solvates, hydrates, mixtures thereof and the salts thereof.

A preferred sub-group relates to (R)-enantiomers of the compounds according to the invention of formula (la)

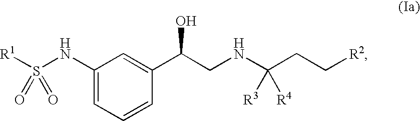

wherein R¹ to R⁴ are as hereinbefore defined, and the salts thereof.

A second preferred sub-group relates to the (S)-enantiomer of the compounds according to the invention of formula (Ib)

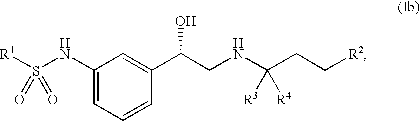

wherein R¹ to R⁴ are as hereinbefore defined, and the salts thereof.

Particular mention should be made of the following compounds:

N-(3-{2-[3-(6-amino-benzimidazol-1-yl)-1,1-dimethyl-propylamino]-1-hydroxy-ethyl}-phenyl)-benzenesulphonamide,

ethyl 1-{3-[2-(3-benzenesulphonylamino-phenyl)-2-hydroxy-ethylamino]-3-methyl-butyl}-1H-benzimidazole-6-carboxylate,

1-{3-[2-(3-benzenesulphonylamino-phenyl)-2-hydroxy-ethylamino]-3-methyl-butyl}-1H-benzimidazole-6-carboxylic acid,

(2-morpholino-ethyl)1-{3-[2-(3-benzenesulphonylamino-phenyl)-2-hydroxy-ethylamino]-3-methyl-butyl}-1H-benzimidazole-5-carboxylate,

1-{3-[2-(3-benzenesulphonylamino-phenyl)-2-hydroxy-ethylamino]-3-methyl-butyl-1H-benzimidazole-5-carboxylic acid hydrazide,

1-{3-[2-(3-benzenesulphonylamino-phenyl)-2-hydroxy-ethylamino]-3-methyl-butyl}-6-methoxy-1H-benzimidazole-5-carboxylic acid,

1-{3-[2-(3-benzenesulphonylamino-phenyl)-2-hydroxy-ethylamino]-3-methyl-butyl}-6-chloro-1H-benzimidazole-5-carboxylic acid,

1-{3-[2-(3-benzenesulphonylamino-phenyl)-2-hydroxy-ethylamino]-3-methyl-butyl}-5-chloro-1H-benzimidazole-6-carboxylic acid,

1-{3-[2-(3-benzenesulphonylamino-phenyl)-2-hydroxy-ethylamino]-3-methyl-butyl}-4-chloro-1H-benzimidazole-5-carboxylic acid,

1-{3-[2-(3-benzenesulphonylamino-phenyl)-2-hydroxy-ethylamino]-3-methyl-butyl-7-chloro-1H-benzimidazole-6-carboxylic acid and

1-{3-[2-(3-benzenesulphonylamino-phenyl)-2-hydroxy-ethylamino]-3-methyl-butyl}-6-hydroxy-1H-benzimidazole-5-carboxylic acid

and the enantiomers and salts thereof,

particularly the compounds:

N-(3-{2-[3-(6-amino-benzimidazol-1-yl)-1,1-dimethyl-propylamino]-1-hydroxy-ethyl}-phenyl)-benzenesulphonamide,

ethyl(R)-1-{3-[2-(3-benzenesulphonylamino-phenyl)-2-hydroxy-ethylamino]-3-methyl-butyl}-1H-benzimidazole-6-carboxylate,

(R)-1-{3-[2-(3-benzenesulphonylamino-phenyl)-2-hydroxy-ethylamino]-3-methyl-butyl}-1H-benzimidazole-6-carboxylic acid,

(2-morpholino-ethyl)(R)-1-{3-[2-(3-benzenesulphonylamino-phenyl)-2-hydroxy-ethylamino]-3-methyl-butyl}-1H-benzimidazole-5-carboxylate,

(R)-1-{3-[2-(3-benzenesulphonylamino-phenyl)-2-hydroxy-ethylamino]-3-methyl-butyl}-1H-benzimidazole-5-carboxylic acid hydrazide,

1-{3-[(R)-2-(3-benzenesulphonylamino-phenyl)-2-hydroxy-ethylamino]-3-methyl-butyl}-6-methoxy-1H-benzimidazole-5-carboxylic acid,

1-{3-[(R)-2-(3-benzenesulphonylamino-phenyl)-2-hydroxy-ethylamino]-3-methyl-butyl}-6-chloro-1H-benzimidazole-5-carboxylic acid,

1-{3-[(R)-2-(3-benzenesulphonylamino-phenyl)-2-hydroxy-ethylamino]-3-methyl-butyl}-5-chloro-1H-benzimidazole-6-carboxylic acid,

1-{3-[(R)-2-(3-benzenesulphonylamino-phenyl)-2-hydroxy-ethylamino]-3-methyl-butyl}-4-chloro-1H-benzimidazole-5-carboxylic acid,

1-{3-[(R)-2-(3-benzenesulphonylamino-phenyl)-2-hydroxy-ethylamino]-3-methyl-butyl}-7-chloro-1H-benzimidazole-6-carboxylic acid and

1-{3-[(R)-2-(3-benzenesulphonylamino-phenyl)-2-hydroxy-ethylamino]-3-methyl-butyl}-6-hydroxy-1H-benzimidazole-5-carboxylic acid

and the enantiomers and salts thereof.

In another aspect the invention relates to the compounds

ethyl(R)-1-{3-[2-(3-benzenesulphonylamino-phenyl)-2-hydroxy-ethylamino]-3-methyl-butyl}-1H-benzimidazole-5-carboxylate and

(R)-1-{3-[2-(3-benzenesulphonylamino-phenyl)-2-hydroxy-ethylamino]-3-methyl-butyl}-1H-benzimidazole-5-carboxylic acid

and the salts thereof.

Another sub-group of the invention relates to compounds of general formula (I), wherein

R¹ denotes a C₁₋₄-alkyl, di-(C₁₋₃-alkyl)-amino, thienyl, pyridyl or phenyl group,
wherein the phenyl group may be substituted by one to three fluorine, chlorine or bromine atoms or one to three C₁₋₃-alkyl, C₁₋₃-alkyloxy, trifluoromethoxy or difluoromethoxy groups, wherein the substituents may be identical or different

R² denotes a benzimidazolyl or 1,3-dihydrobenzimidazol-2-one group,
each of which may be substituted by one or two fluorine, chlorine or bromine atoms or a C₁₋₃-alkyl, methoxy, trifluoromethoxy, difluoromethoxy, carboxy, C₁₋₄-alkyloxy-carbonyl or amino group, wherein the substituents may be identical or different or
wherein two adjacent carbon atoms may be bridged by a —CH═CH—CH═CH— group,

and R³ and R⁴, which may be identical or different, each denote a C₁₋₃-alkyl group,

while the alkyl groups contained in the above-mentioned groups may be straight-chain or branched,

with the exclusion of the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl}-1H-benzimidazole-5-carboxylic acid and

ethyl 1-{3-[2-(3-benzenesulphonyamino-phenyl)-2-hydroxy-ethylamino]-3-methyl-butyl}-1H-benzimidazole-5-carboxylate;

optionally in the form of the tautomers, racemates, enantiomers, diastereomers, solvates and hydrates and mixtures thereof, and optionally the prodrugs, double prodrugs and salts thereof, particularly the physiologically acceptable salts thereof with inorganic or organic acids or bases.

Another preferred sub-group comprises those compounds of general formula (I), wherein

R², R³ and R⁴ are as hereinbefore defined and

R¹ denotes a phenyl group, which may be substituted by a fluorine, chlorine or bromine atom or a C₁₋₃-alkyl, C₁₋₃-alkyloxy, trifluoromethoxy or difluoromethoxy groups,

excluding the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl-1H-benzimidazole-5-carboxylic acid and

ethyl 1-{3-[2-(3-benzenesulphonylamino-phenyl)-2-hydroxy-ethylamino]-3-methyl-butyl}-1H-benzimidazole-5-carboxylate;

and the tautomers, racemates, enantiomers, diastereomers, solvates, hydrates, mixtures thereof and the salts thereof,

particularly those compounds of general formula (I), wherein

R² is as hereinbefore defined,

R¹ denotes a phenyl group

and R³ and R⁴ each represent a methyl group,

while excluding the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl}-1H-benzimidazole-5-carboxylic acid and

ethyl 1-{3-[2-(3-benzenesulphonyamino-phenyl)-2-hydroxy-ethylamino]-3-methyl-butyl}-1H-benzimidazole-5-carboxylate;

the tautomers, enantiomers, diastereomers, mixtures thereof and the salts thereof.

A particularly preferred sub-group comprises those compounds of general formula (I), wherein

R¹ denotes a phenyl group,

R² denotes a benzimidazol-1-yl or 1,3-dihydrobenzimidazol-2-on-1-yl group,
each of which may be substituted by one or two fluorine, chlorine or bromine atoms or a C₁₋₃-alkyl, carboxy, C₁₋₄-alkyloxy-carbonyl or amino group or
wherein two adjacent carbon atoms may be bridged by a —CH═CH—CH═CH— group,

and R³ and R⁴ each represent a methyl group,

while the alkyl groups contained in the above-mentioned groups may be straight-chain or branched,

and excluding the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl}-1H-benzimidazole-5-carboxylic acid and

ethyl 1-{3-[2-(3-benzenesulphonyamino-phenyl)-2-hydroxy-ethylamino]-3-methyl-butyl}-1H-benzimidazole-5-carboxylate;

the tautomers, enantiomers, diastereomers, mixtures thereof and the salts thereof.

A most particularly preferred sub-group comprises those compounds of general formula (I), wherein

R² denotes a benzimidazol-1-yl group,
which may be substituted by one or two fluorine, chlorine or bromine atoms or a C₁₋₃-alkyl, methoxy, trifluoromethoxy, difluoromethoxy, carboxy, C₁₋₄-alkyloxy-carbonyl or amino group, wherein the substituents may be identical or different or
wherein two adjacent carbon atoms may be bridged by a —CH═CH—CH═CH— group,

while the alkyl groups contained in the above-mentioned groups may be straight-chain or branched,

and excluding the compounds

N-(3-{2-[3-(5-amino-benzimidazol-1-yl)-1,1-dimethyl-propylamino]-1-hydroxy-ethyl}-phenyl)-benzenesulphonamide,

1-{3-[2-(3-benzenesulphonylamino-phenyl)-2-hydroxy-ethylamino]-3-methyl-butyl}-1H-benzimidazole-5-carboxylic acid and

ethyl 1-{3-[2-(3-benzenesulphonylamino-phenyl)-2-hydroxy-ethylamino]-3-methyl-butyl}-1H-benzimidazole-5-carboxylate;

the tautomers, enantiomers, diastereomers, mixtures thereof, the prodrugs thereof and the salts thereof.

The invention also relates to compounds of general formula (I) for use as pharmaceutical compositions.

The invention also relates to compounds of general formula (I) for use as pharmaceutical compositions with a selective beta-3-agonistic activity.

The invention also relates to compounds of general formula (I) for preparing a pharmaceutical composition for the treatment and/or prevention of diseases connected with the stimulation of beta-3-receptors.

The invention further relates to a method for the treatment and/or prevention of diseases connected with the stimulation of beta-3-receptors, in which a patient is given an effective amount of a compound of general formula I.

The invention further relates to a pharmaceutical composition containing as active substance one or more compounds of general formula (I), optionally combined with conventional excipients and/or carriers.

The invention further relates to a pharmaceutical composition containing as active substance one or more compounds of general formula (I) or the physiologically acceptable salts thereof and one or more active substances selected from among antidiabetics, inhibitors of protein tyrosinephosphatase 1, substances which influence deregulated glucose production in the liver, lipid lowering agents, cholesterol absorption inhibitors, HDL-raising compounds, active substances for the treatment of obesity and modulators or stimulators of the adrenergic system via alpha 1 and alpha 2 as well as beta 1, beta 2 and beta 3 receptors.

The invention also relates to a process for preparing a compound of general formula

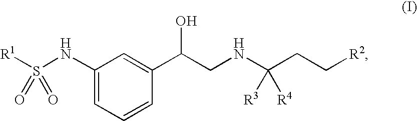

wherein

R¹ to R⁴ may have the meanings given hereinbefore,

wherein a compound of general formula (II)

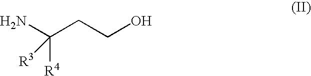

wherein

R³ and R⁴ may have the meaning given hereinbefore,

is converted by means of a chlorinating agent into a compound of formula (III)

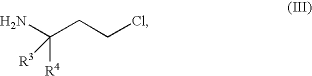

the compound of formula (III) or alternatively a compound of formula (VII)

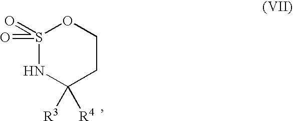

wherein R³ and R⁴ have the meaning given hereinbefore,

each optionally provided with an amino protecting group, is reacted with a compound of formula

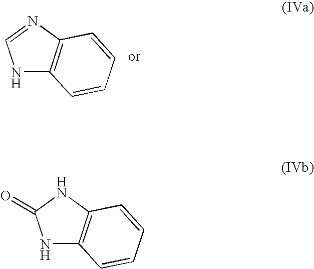

each of which may be substituted by one or two fluorine, chlorine or bromine atoms or one or two C₁₋₃-alkyl, hydroxy, methoxy, trifluoromethoxy, difluoromethoxy, carboxy, C₁₋₄-alkyloxy-carbonyl, ω-morpholin-4-yl-C₂₋₄-alkyloxy-carbonyl, hydrazinocarbonyl or amino groups, wherein the substituents may be identical or different or

wherein two adjacent carbon atoms may be bridged by a —CH═CH—CH═CH— group,

and the product thus obtained of formula (V)

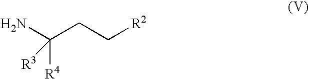

wherein R², R³ and R⁴ have the meanings given hereinbefore,

is reacted with a compound of formula (VIa), (VIb) or (VIc)

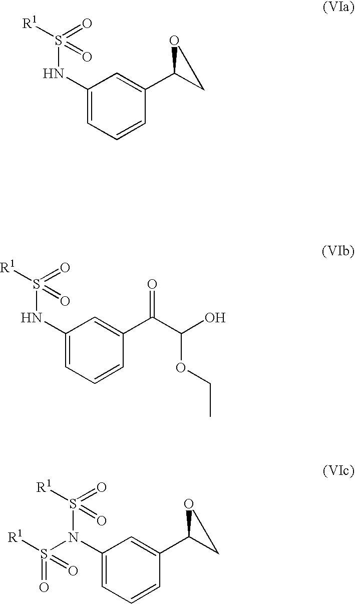

wherein R¹ has the meaning given hereinbefore,

while if (V) is reacted with (VIc) the disulphonamide is subsequently saponified to obtain the monosulphonamide, and then separation of the enantiomers is optionally carried out.

The reaction with the compound (VIb) leads to the racemate, whereas the synthesis with the compound (VIa) or (VIc) yields the respective (R)-enantiomer. An analogous reaction with the enantiomer to (VIa) or (VIc), leading to the (S)-enantiomer, is, of course, also conceivable.

The term alkyl groups, including alkyl groups which are a part of other groups, unless otherwise stated, denotes branched and unbranched alkyl groups with 1 to 10 carbon atoms, while groups with 1 to 6 carbon atoms are preferred. Particularly preferred are alkyl groups with 1 to 4 carbon atoms, particularly those with 1 or 2 carbon atoms. Examples include: methyl, ethyl, propyl, butyl, pentyl, hexyl, heptyl, octyl, nonyl and decyl. Unless otherwise stated, the above-mentioned terms propyl, butyl, pentyl, hexyl, heptyl, octyl, nonyl and decyl include all the possible isomeric forms. For example, the term propyl includes the two isomeric groups n-propyl and iso-propyl, the term butyl includes n-butyl, iso-butyl, sec. butyl and tert.-butyl, the term pentyl includes iso-pentyl, neopentyl, etc.

In the above-mentioned alkyl groups one or more hydrogen atoms may optionally be replaced by other groups. For example, these alkyl groups may be substituted by the halogen atoms fluorine, chlorine, bromine or iodine. Preferably the substituents are fluorine or chlorine. The substituent fluorine is most preferred. All the hydrogen atoms of the alkyl group may optionally also be replaced.

Similarly, in the above-mentioned alkyl groups, unless otherwise stated, one or more hydrogen atoms may optionally be replaced, for example, by OH, NO₂, CN or an optionally substituted group selected from among —O—C₁-C₅-alkyl, preferably methoxy or ethoxy, —O—(C₆-C₁₄-aryl), preferably phenyloxy, —O-heteroaryl, preferably —O-thienyl, —O-thiazolyl, —O-imidazolyl, —O-pyridyl, —O-pyrimidyl or —O-pyrazinyl, saturated or unsaturated —O-heterocycloalkyl, preferably —O-pyrazolyl, —O-pyrrolidinyl, —O-piperidinyl, —O-piperazinyl or —O-tetrahydro-oxazinyl, C₆-C₁₄-aryl, preferably phenyl, heteroaryl, preferably thienyl, thiazolyl, imidazolyl, pyridyl, pyrimidyl or pyrazinyl, saturated or unsaturated heterocycloalkyl, preferably pyrazolyl, pyrrolidinyl, piperidinyl, piperazinyl or tetrahydro-oxazinyl, an amine group, preferably methylamine, benzylamine, phenylamine or heteroarylamine, saturated or unsaturated bicyclic ring systems, preferably benzimidazolyl and C₃-C₈-cycloalkyl, preferably cyclohexyl or cyclopropyl.

Alkenyl groups as well as alkenyl groups which are a part of other groups denote branched and unbranched alkyl groups with 1 to 10 carbon atoms, preferably 1 to 6, particularly preferably 1 to 4 carbon atoms, which contain at least one carbon-carbon double bond. Examples include: ethenyl, propenyl, methylpropenyl, butenyl, pentenyl, hexenyl, heptenyl, methylheptenyl, octenyl, nonenyl and decenyl. Unless stated otherwise, the terms propenyl, butenyl, pentenyl, hexenyl, heptenyl, octenyl, nonenyl and decenyl used above include all the possible isomeric forms. For example, the term butenyl includes the isomeric groups but-1-enyl, but-2-enyl and but-3-enyl, etc.

In the above-mentioned alkenyl groups one or more hydrogen atoms may optionally be replaced by other groups. For example, these alkenyl groups may be substituted by the halogen atoms fluorine, chlorine, bromine or iodine. The substituents fluorine or chlorine are preferred. The substituent fluorine is particularly preferred. It is also possible to replace all the hydrogen atoms of the alkenyl group.

Alkynyl groups as well as alkynyl groups which are a part of other groups denote branched and unbranched alkyl groups with 1 to 10 carbon atoms, preferably 1 to 6, particularly preferably 1 to 4 carbon atoms which contain at least one carbon-carbon triple bond. Examples include: ethynyl, propynyl, butynyl, pentynyl, hexynyl, heptynyl, octynyl, nonynyl and decynyl. Unless otherwise mentioned, the terms propynyl, butynyl, pentynyl, hexynyl, heptynyl, octynyl, nonynyl and decynyl used above include all the possible isomeric forms. For example, the term butynyl includes the isomeric groups but-1 -ynyl, but-2-ynyl and but-3-ynyl, etc. In the above-mentioned alkynyl groups one or more hydrogen atoms may optionally be replaced by other groups. For example, these alkynyl groups may be substituted by the halogen atoms fluorine, chlorine, bromine or iodine. The substituents fluorine or chlorine are preferred. The substituent fluorine is particularly preferred. It is also possible to replace all the hydrogen atoms of the alkynyl group.

The term aryl denotes an aromatic ring system with 6 to 18 carbon atoms, preferably 6 to 14 carbon atoms, preferably 6 or 10 carbon atoms, most preferably phenyl, which may optionally be substituted and may preferably carry one or more of the following substituents: OH, NO₂, CN, —OCHF₂, —OCF₃, —NH₂, —NH-alkyl, —N(alkyl)-alkyl, —NH-aryl, —N(alkyl)-aryl, —NHCO-alkyl, —NHCO-aryl, —N(alkyl)-CO-alkyl, —N(alkyl)-CO-aryl, —NHSO₂-alkyl, —NHSO₂—N(alkyl)₂, —NHSO₂-aryl, —N(alkyl)-SO₂-alkyl, —N(alkyl)-SO₂-aryl, —CO₂-alkyl, —SO₂-alkyl, —SO₂-aryl, —CONH(OH), —CONH-alkyl, —CONH-aryl, —CON(alkyl)-alkyl, —CON(alkyl)-aryl, —SO₂NH-alkyl, —SO₂NH-aryl, —SO₂N(alkyl)-alkyl, —SO₂N(alkyl)-aryl, —O-alkyl, —O-aryl —S-alkyl, —S-aryl, tetrazolyl, halogen, for example fluorine, chlorine, bromine or iodine, preferably fluorine or chlorine, particularly fluorine, C₁-C₁₀-alkyl, preferably C₁-C₅-alkyl, particularly preferably C₁-C₃-alkyl, most particularly preferably methyl or ethyl, —O—(C₁-C₃-alkyl), preferably methoxy or ethoxy, —COOH or —CONH₂.

Examples of heteroaryl groups are 5- to 10-membered mono- or bicyclic heteroaryl rings wherein one to three carbon atoms in each case may be replaced by a heteroatom selected from among oxygen, nitrogen or sulphur. Examples include furan, thiophene, pyrrole, pyrazole, imidazole, triazole, tetrazole, pyridine, pyridazine, pyrimidine, pyrazine, triazine, oxazole, isoxazole, thiazole, thiadiazole, oxadiazole, while each of the above-mentioned heterocycles may optionally also be annellated to a benzene ring, such as benzimidazole, and these heterocycles may optionally be substituted and preferably carry one or more of the following substituents: OH, NO₂, CN, —NH₂, —NH-alkyl, —N(alkyl)-alkyl, —NH-aryl, —N(alkyl)-aryl, —NHCO-alkyl, —NHCO-aryl, —N(alkyl)-CO-alkyl, —N(alkyl)-CO-aryl, —NHSO₂-alkyl, —NHSO₂-aryl, —N(alkyl)-SO₂-alkyl, —N(alkyl)-SO₂-aryl, —CO₂-alkyl, —SO₂-alkyl, —SO₂-aryl, —CONH-alkyl, —CONH-aryl, —CON(alkyl)-alkyl, —CON(alkyl)-aryl, —SO₂NH-alkyl, —SO₂NH-aryl, —SO₂N(alkyl)-alkyl, —SO₂N(alkyl)-aryl, —O-alkyl, —O-aryl —S-alkyl, —S-aryl, —CONH₂, halogen, preferably fluorine or chlorine, C₁-C₁₀-alkyl, preferably C₁-C₅-alkyl, preferably C₁-C₃-alkyl, particularly preferably methyl or ethyl, —O—(C₁-C₃-alkyl), preferably methoxy or ethoxy, —COOH, —COOCH₃, —CONH₂, —SO-alkyl, —SO₂-alkyl, —SO₂H, —SO₃-alkyl or optionally substituted phenyl.

Examples of cycloalkyl groups are saturated or unsaturated cycloalkyl groups with 3 to 8 carbon atoms, for example cyclopropyl, cyclobutyl, cyclopentyl, cyclopentenyl, cyclohexyl, cyclohexenyl, cycloheptyl or cyclooctyl, preferably cyclopropyl, cyclopentyl or cyclohexyl, while each of the above-mentioned cycloalkyl groups may optionally also carry one or more substituents or be annellated to a benzene ring.

Unless otherwise stated in the definitions, examples of heterocycloalkyl or heterocyclyl groups include 5- , 6- or 7-membered, saturated or unsaturated heterocycles which may contain nitrogen, oxygen or sulphur as heteroatoms, for example tetrahydrofuran, tetrahydrofuranone, γ-butyrolactone, α-pyran, γ-pyran, dioxolane, tetrahydropyran, dioxane, dihydrothiophene, thiolane, dithiolane, pyrroline, pyrrolidine, pyrazoline, pyrazolidine, imidazoline, imidazolidine, tetrazole, piperidine, pyridazine, pyrimidine, pyrazine, piperazine, triazine, tetrazine, morpholine, thiomorpholine, diazepan, oxazine, tetrahydro-oxazinyl, isothiazole and pyrazolidine, preferably pyrazolyl, pyrrolidinyl, piperidinyl, piperazinyl or tetrahydro-oxazinyl, while the heterocyclic group may optionally be substituted.

The compounds of the above general formula (I) which contain a group that can be cleaved in-vivo are so-called prodrugs, and compounds of general formula I which contain two groups that can be cleaved in-vivo are so-called double prodrugs.

By a group which can be converted in-vivo into a carboxy group is meant for example an ester of formula —CO₂R¹¹, where

R¹¹ denotes hydroxymethyl, alkenyl, alkynyl, aryl, heteroaryl, cycloalkenyl, hetero-cycloalkyl, C₁-C₃-alkoxycarbonyl, 1,3-dihydro-3-oxo-1-isobenzofuranol, —C(-alkyl)(-alkyl)-OC(O)-alkyl, —CHC(O)NH(-alkyl), —CHC(O)N(-alkyl)(-alkyl), -alkyl, preferably C₁-C₆-alkyl, particularly preferably methyl, ethyl, n-propyl, isopropyl, n-butyl, n-pentyl or n-hexyl,

cycloalkyl, preferably C₁-C₆-cycloalkyl, particularly preferably cyclohexyl, —(C₁-C₃-alkyl)-aryl, preferably (C₁-C₃-alkyl)-phenyl, particularly preferably benzyl, —CHC(O)N(-alkyl)(-alkyl), preferably —CHC(O)N(—C₁-C₃-alkyl)(—C₁-C₃-alkyl), particularly preferably —CHC(O)N(CH₃)₂,

—CH(-alkyl)OC(O)-alkyl, preferably —CH(—CH₃)OC(O)(—C₁-C₆-alkyl), particularly preferably —CH(—CH₃)OC(O)-methyl, —CH(—CH₃)OC(O)-ethyl, —CH(—CH₃)OC(O)-n-propyl, —CH(—CH₃)OC(O)-n-butyl or —CH(—CH₃)OC(O)-t-butyl, or

—CH₂OC(O)-alkyl, preferably —CH₂OC(O)(—C₁-C₆-alkyl), particularly preferably —CH₂OC(O)-methyl, —CH₂OC(O)-ethyl, —CH₂OC(O)-n-propyl, —CH₂OC(O)-n-butyl or —CH₂OC(O)-t-butyl.

By a group which can be converted in-vivo into a sulphonamide or amino group is meant for example one of the following groups:

—OH, -formyl, —C(O)-alkyl, —C(O)-aryl, —C(O)-heteroaryl, —CH₂OC(O)-alkyl,

—CH(-alkyl)OC(O)-alkyl, —C(-alkyl)(-alkyl)OC(O)-alkyl,

—CO₂-alkyl, preferably C₁-C₉-alkoxy-carbonyl, particularly preferably methoxycarbonyl, ethoxycarbonyl, n-propyloxycarbonyl, isopropyloxycarbonyl, n-butyloxycarbonyl, n-pentyloxycarbonyl, n-hexyloxycarbonyl, cyclohexyloxycarbonyl, n-heptyloxycarbonyl, n-octyloxycarbonyl or n-nonyloxycarbonyl,

—CO₂(—C₁-C₃-alkyl)-aryl, preferably —CO₂(—C₁-C₃-alkyl)-phenyl, particularly preferably benzyloxycarbonyl,

—C(O)-aryl, preferably benzoyl,

—C(O)-heteroaryl, preferably pyridinoyl or nicotinoyl or

—C(O)-alkyl, preferably —C(O)(—C₁-C₆-alkyl), particularly preferably 2-methylsulphonyl-ethoxycarbonyl, 2-(2-ethoxy)-ethoxycarbonyl.

Halogen generally denotes fluorine, chlorine, bromine or iodine, preferably chlorine or fluorine, particularly preferably fluorine.

The compounds according to the invention may be in the form of the individual optical isomers, mixtures of the individual enantiomers, diastereomers or racemates, prodrugs, double prodrugs and in the form of the tautomers, salts, solvates and hydrates as well as in the form of the free bases or the corresponding acid addition salts with pharmacologically acceptable acids—such as for example acid addition salts with hydrohalic acids, for example hydrochloric or hydrobromic acid, or organic acids, such as for example oxalic, fumaric, diglycolic, formic, malic, benzoic, benzenesulphonic, camphorsulphonic, acetic, ethanesulphonic, glutamic, maleic, mandelic, lactic, phosphoric, nitric, sulphuric, succinic, para-toluenesulphonic, trifluoroacetic, tartaric, citric or methanesulphonic acid.

Moreover, if the new compounds of formula I thus obtained contain a carboxy group or another acid group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

Moreover the compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be, for example, (+) or (−)-menthol and an optically active acyl group in amides, for example, may be a (+)- or (−)-menthyloxycarbonyl.

As has been found, the compounds of general formula (I) are characterised by their great versatility in the therapeutic field. Particular mention should be made of those applications in which the effects of beta-3-agonists, particularly selective beta-3-agonists play a part.

Such diseases include for example:

atherosclerosis, cholangitis, gall bladder disease, chronic cystitis, chronic bladder inflammation; chronic prostatitis, cystospasm, depression, duodenal ulcer, duodenitis, dysmenorrhoea; increased intraocular pressure and glaucoma, enteritis, oesophagitis, gastric ulcer, gastritis, gastrointestinal disorders caused by contraction(s) of the smooth muscle, gastrointestinal disorders incl. gastric ulcer; gastrointestinal ulceration, gastrointestinal ulcers, glaucoma, glucosuria, hyperanakinesia, hypercholesterolaemia, hyperglycaemia, hyperlipaemia, arterial hypertension, hypertriglyceridaemia, insulin resistance, intestinal ulceration or small bowel ulcers (incl. inflammatory bowel diseases, ulcerative colitis, Crohn's disease and proctitis=inflammation of the rectum), irritable colon and other diseases with decreased intestinal motility, depression, melancholy, pollacisuria, frequent urinary urgency, nervous neurogenic inflammation, neurogenic bladder dysfunction, neurogenic inflammation of the respiratory tract, neuropathic bladder dysfunction, nycturia, non-specific diarrhoea, dumping syndrome, obesity, fatness, pancreatitis, inflammation of the pancreas, stomach ulcers, prostate diseases such as benign prostatic hyperplasia, enlarged prostate, spasm, cramp, type 2 diabetes mellitus, irritable bladder or concrement of the lower urinary tract.

The following may also be mentioned: urge incontinence, stress incontinence, mixed incontinence, overactive bladder (OAB) in the forms of wet OAB or dry OAB, OAB with imperative need to urinate, with or without urge incontinence, with or without increased frequency of urination, with or without nocturnal urination, dysuria, nycturia, pollacisuria, build-up of residual urine. Of these indications, OAB with increased frequency of urination, with or without urge incontinence, with or without nocturnal urination, is preferred.

The compounds may also be used in cases of pain in the prostate or of the lower urogenital tract. The diseases in question include benign prostatic hyperplasiam (BPH), prostatitis, particularly chronic abacterial prostatitis, of neurogenic, muscular or bacterial origin, chronic pain syndrome of the pelvis, pelvic myoneuropathy, prostatodynia, LUTS (lower urinary tract symptoms), obstructive bladder emptying disorders (BOO) and/or prostatopathy.

The use according to the invention is directed not only to causative treatment of the above indications, but also to the treatment of the accompanying symptoms, particularly any related pain or problems of urine release, pain and discomfort in the region of the prostate or the lower urinary tract including the penis, pain during erection or ejaculation, pain on defecation, erectile disorders.

The compounds according to the invention are also suitable for the treatment of neurodegenerative diseases such as e.g. Alzheimer's disease, Parkinson's disease or Huntington's disease.

The beta-3 agonists according to the invention are particularly suitable for the treatment of obesity, insulin resistance, type 2 diabetes mellitus, urinary incontinence, irritable colon and other diseases with decreased intestinal motility or depression, particularly for the treatment of diabetes and obesity.

The activity of the beta-3 agonists can be determined for example in a lipolysis test. The test procedure may be carried out as follows:

Adipocytes were isolated from fatty tissue ex vivo by modifying a method according to Rodbell (Rodbell, M. Metabolism of isolated fat cells. I. Effects of hormones on glucose metabolism and lipolysis. J Biol Chem 239: 375-380. 1964). The excised fatty tissue was cut into small pieces and mixed with 1 mg/ml collagenase in Krebs Ringer Buffer (KRB) containing 6 mM glucose and 2% albumin by gently shaking for 30-40 min at 37° C. The cells were filtered through a gauze, washed twice with KRB and in each case 50-150 g were centrifuged for 5 min. 10 μl of the centrifuged adipocytes were incubated with 90 μl of a compound according to the invention (agonist) at concentrations of between 10⁻¹⁵ to 10⁻⁴ M. The agonists were incubated over 40 min at 37° C. A varying release of glycerol into the medium indicated that the fat cell lipolysis had altered as a result of the addition of the agonist. Released glycerol was detected enzymatically with a Sigma kit (triglyceride (GPO Trinder) Reagent A; Cat. #337-40A), as described below.

Glycerol is phosphorylated by ATP via glycerol kinase. The resulting glycerol-1-phosphate is oxidised by glycerolphosphate oxidase to form dihydroxyacetone phosphate and hydrogen peroxide. Then a quinonimine dye is produced by the peroxidase-catalysed coupling of sodium- N-ethyl-N-(3-sulphopropyl)m-ansidine and 4-aminoantipyrine. The dye has an absorption peak at 540 nm. The absorption is directly proportional to the glycerol concentration in the samples.

The new compounds may be used for the prevention or short-term or long-term treatment of the above-mentioned diseases, and may also be used in conjunction with other active substances used for the same indications. These include, for example, antidiabetics, such as metformin, sulphonylureas (e.g. glibenclamid, tolbutamide, glimepiride), nateglinide, repaglinide, thiazolidinedione (e.g. rosiglitazone, pioglitazone), PPAR-gamma agonists (e.g. GI 262570), alpha-gluco-sidase inhibitors (e.g. acarbose, voglibose), alpha2 antagonists, insulin and insulin analogues, GLP-1 and GLP-1 analogues (e.g. exendin-4) or amylin. Also, inhibitors of protein tyrosine phosphatase 1, substances which influence deregulated glucose production in the liver, such as e.g. inhibitors of glucose-6-phosphatase, or fructose-1,6-bisphosphatase, glycogen phosphorylase, glucagon receptor antagonists and inhibitors of phosphoenol pyruvate carboxykinase, glycogen synthase kinase or pyruvate dehydrokinase, lipid lowering agents, such as HMG-CoA-reductase inhibitors (e.g. simvastatin, atorvastatin), fibrates (e.g. bezafibrate, fenofibrate), nicotinic acid and its derivatives, cholesterol absorption inhibitors such as for example ezetimibe, bile acid-binding substances such as for example cholestyramine, HDL-raising compounds such as for example inhibitors of CETP or regulators of ABC1 or active substances for the treatment of obesity, such as e.g. sibutramine or tetrahydrolipostatin.

In particular, they may also be combined with drugs for treating high blood pressure such as e.g. All antagonists or ACE inhibitors, diuretics, β-blockers, and other modulators of the adrenergic system or combinations thereof. In addition, combinations with stimulators of the adrenergic system via alpha 1 and alpha 2 and also beta 1, beta 2 and beta 3 receptors are particularly suitable.

The compounds of general formula (I) may be used on their own or in conjunction with other active substances according to the invention, optionally also in conjunction with other pharmacologically active substances. Suitable preparations include for example tablets, capsules, suppositories, solutions, particularly solutions for injection (s.c., i.v., i.m.) and infusion, elixirs, emulsions or dispersible powders. The content of the pharmaceutically active compound(s) should be in the range from 0.1 to 90 wt. %, preferably 0.5 to 50 wt. % of the composition as a whole, i.e. in amounts which are sufficient to achieve the dosage range specified below. The specified doses may be taken several times a day, if necessary.

Suitable tablets may be obtained, for example, by mixing the active substance(s) with known excipients, for example inert diluents such as calcium carbonate, calcium phosphate or lactose, disintegrates such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, optionally organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, preferably oral. For oral administration the tablets may, of course contain, apart from the above-mentioned carriers, additives such as sodium citrate, calcium carbonate and dicalcium phosphate together with various added substances such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded.

When administering large amounts it may be advisable to divide them up into a number of smaller doses spread over the day.

The formulation Examples which follow illustrate the present invention without restricting its scope:

Examples of pharmaceutical formulations

| A) Tablets | per tablet |
|---|---|
| active substance | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) Tablets | per tablet |
|---|---|
| active substance | 80 mg |
| lactose | 55 mg |
| corn starch | 190 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| C) Ampoule solution | | |
|---|---|---|
| active substance | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

The following Examples are intended to illustrate the invention without restricting its scope.

Analytical HPLC Method:

Retention times were determined using an apparatus made by Agilent, type 1100 (quaternary pump, diode array detector, LC-MSD) fitted with a Merck Cromolith Speed ROD column (RP18e, 50×4.6 mm). For elution mixtures of acetonitrile and water, in each case modified with 0.1% formic acid, were used at a flow rate of 1.5 ml/min with the following gradient pattern:

| time [minutes] | vol % water |
|---|---|
| 0.0 | 10 |
| 4.5 | 90 |
| 5.0 | 90 |
| 5.5 | 10 |

Preparation of the Starting Compounds

### Component 1: tert-butyl (3-chloro-1,1-dimethyl-propyl)-carbamate

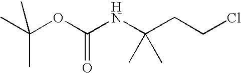

### Step 1: 3-chloro-1,1-dimethylpropylamine-hydrochloride

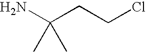

48.7 mL (668 mmol) thionyl chloride are slowly added dropwise at 0° C. to a solution of 53.0 g (514 mmol) 3-amino-3-methyl-butanol in 255 mL dichloromethane/dimethylformamide (50/1). After the addition has ended the reaction mixture is refluxed for 1 hour (h) and then stirred for 16 h at ambient temperature. The reaction mixture is evaporated down using the rotary evaporator and the residue is combined with 50 mL acetonitrile with stirring. The solid is filtered off and dried for 18 h at 45° C. dried. 67.9 g (430 mmol, 84%) 3-chloro-1,1-dimethylpropylamine-hydrochloride are obtained as a colourless solid.

R_{f}=0.52 [silica gel, dichloromethane/methanol/ammonia (90/9/1)]

MS [ESI (M+H)⁺]=122/4 (Cl)

### Step 2: tert-butyl (3-chloro-1,1-dimethyl-propyl)-carbamate

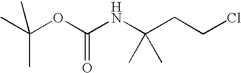

101 g (218 mmol) di-tert.-butyldicarbonate are added batchwise at ambient temperature to a solution of 48.8 g (309 mmol) 3-chloro-1,1-dimethylpropylamine-hydrochloride and 100 mL (718 mmol) triethylamine in 900 mL dichloromethane. After the addition has ended the reaction mixture is stirred for 4 days at RT. The reaction mixture is evaporated down using the rotary evaporator and the residue is taken up in 250 mL ethyl acetate and 400 mL water. The phases are separated and the aqueous phase is extracted with ethyl acetate. The combined organic phases are washed with water, dried on sodium sulphate and concentrated by evaporation in the rotary evaporator. 45.3 g (204 mmol, 66 %) tert-butyl (3-chloro-1,1-dimethyl-propyl)-carbamate are obtained as a colourless oil.

R_{f}=0.90 [silica gel, dichloromethane/methanol (90/1)]

MS [ESI (M+H)⁺]=222/4 (Cl)

### Component 2: N-(3-acetyl-phenyl)-benzenesulphonamide

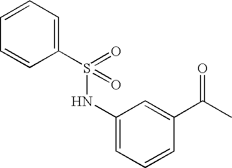

Summa, Vincenzo; Petrocchi, Alessia; Pace, Paola; Matassa, Victor G.; Francesco, Raffaele De; Altamura, Sergio; Tomei, Licia; Koch, Uwe; Neuner, Philippe; J. Med. Chem.; 47; 1; 2004; 14-17.

### Component 3 N-[3-(2-ethoxy-2-hydroxyacetyl)-phenyl]-benzenesulphonamide

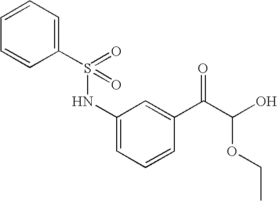

1 mL water, 1 g activated charcoal and 2.66 g (24 mmol) selenium dioxide is added to a solution of 1.65 g (6.00 mmol) N-(acetylphenyl)benzenesulphonamide in about 10 mL dioxane. The reaction mixture is stirred for 4 days at 80° C. and then concentrated by evaporation in the rotary evaporator. The residue is dissolved in about 30 mL ethanol and refluxed for 4 h. The reaction mixture is evaporated down using the rotary evaporator, the residue is dissolved in ethyl acetate, washed with saturated, aqueous sodium hydrogen carbonate solution, dried on sodium sulphate and concentrated by evaporation in the rotary evaporator. 0.917 g (2.73 mmol, 46%) N-[3-(2-ethoxy-2-hydroxyacetyl)-phenyl]-benzenesulphonamide are obtained as a yellow solid.

R_{f}=0.21 [silica gel, petroleum ether/ethyl acetate (1/1)]

### Component 4 (R)—N-(3-oxiranyl-phenyl)-benzenesulphonamide

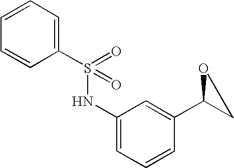

### Step 1: N-[3-(2-chloro-acetyl)-phenyl]-benzenesulphonamide

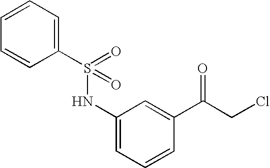

9.81 mL (121 mmol) sulphuryl chloride are added dropwise at 0° C. over 25 minutes (min) with vigorous stirring to 11.1 g (40.3 mmol) N-(3-acetyl-phenyl)-benzenesulphonamide in 200 mL dichloromethane and 9.80 mL (138 mmol) methanol. The reaction mixture is refluxed for 3 h and then cooled to ambient temperature. It is washed successively with water, saturated aqueous sodium hydrogen carbonate solution and saturated aqueous sodium chloride solution. The organic phase is dried and concentrated by evaporation in the rotary evaporator, to obtain 12.5 g (40.3 mmol, quantitative) N-[3-(2-chloro-acetyl)-phenyl]-benzenesulphonamide.

R_{f}=0.38 [silica gel, petroleum ether/ethyl acetate (65/35)]

MS [ESI (M−H)⁻]=308/10 (Cl)

### Step 2: (R)—N-[3-(2-chloro-1-hydroxy-ethyl)-phenyl]-benzenesulphonamide

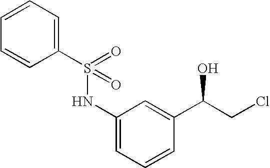

17.8 g (55.4 mmol) (−)-B-chlorodiisopinocamphenylboran [(−)-DIP-chloride] (dissolved in 20 mL tetrahydrofuran) are added dropwise at −30° C. to 5.20 g (16.8 mmol) N-[3-(2-chloro-acetyl)-phenyl]-benzenesulphonamide in tetrahydrofuran. The reaction mixture is stirred for 15 h at this temperature, then poured into ice-cooled, saturated, aqueous sodium hydrogen carbonate solution and extracted with ethyl acetate. The combined organic phases are washed successively with water and saturated, aqueous sodium chloride solution, dried on magnesium sulphate and concentrated by evaporation in the rotary evaporator. The residue is purified by flash column chromatography [silica gel, petroleum ether/ethyl acetate (95:5→60:40)], to obtain 4.40 g (14.1 mmol, 84%) (R)-N-[3-(2-chloro-1-hydroxy-ethyl)-phenyl]-benzenesulphonamide.

R_{f}=0.15 [silica gel, petroleum ether/ethyl acetate (2/1]

MS [ESI (M−H)⁻]=310/12 (Cl)

### Step 3: (R)—N-(3-oxiranyl-phenyl)-benzenesulphonamide

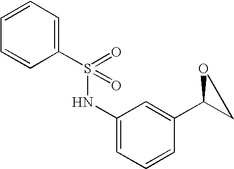

1.25 g (4.00 mmol) (R)—N-[3-(2-chloro-1-hydroxy-ethyl)-phenyl]-benzenesulphonamide in and 1.38 g (10.0 mmol) potassium carbonate are refluxed for 4 h in 30 mL acetonitrile. The reaction mixture is evaporated down using the rotary evaporator, combined with water and extracted with ethyl acetate. The combined organic phases are washed successively with water and saturated, aqueous sodium chloride solution, dried on magnesium sulphate and concentrated by evaporation in the rotary evaporator. The residue is purified by flash column chromatography [silica gel, petroleum ether/ethyl acetate (90:10→50:50)], to obtain 0.80 g (2.91 mmol, 73%) (R)—N-(3-oxiranyl-phenyl)-benzenesulphonamide.

R_{f}=0.38 [silica gel, petroleum ether/ethyl acetate (7/3)]

MS [ESI (M−H)⁻]=310/12 (Cl)

### Component 5 N—[(R)-3-oxiranyl-phenyl]-dibenzenesulphonamide

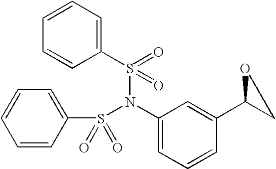

### Step 1: N-(3-acetyl-phenyl)-dibenzenesulphonamide

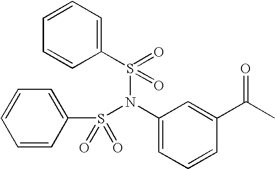

2.75 g (10 mmol) N-(3-acetyl-phenyl)-benzenesulphonamide are dissolved in 50 ml acetonitrile and combined with 3.3 ml (24 mmol) triethylamine. With vigorous stirring 3.89 g (22 mmol) benzenesulphonic acid chloride are added dropwise over a period of 10 minutes at ambient temperature. The reaction mixture is then stirred for 20 hours at ambient temperature and then concentrated by evaporation in the rotary evaporator. The residue is poured into ice water, whereupon a beige solid is precipitated. This precipitate is filtered off and recrystallised from ethyl acetate.

Yield: 3.6 g (87% of theory)

C₂₀H₁₇NO₅S₂ (415.49)

MS [ESI (M+NH₄)⁺]=433

R_{f}=0.44 [silica gel, toluene/ethyl acetate (9/1)]

### Step 2: N-[3-(2-chloro-acetyl)-phenyl]-dibenzenesulphonamide

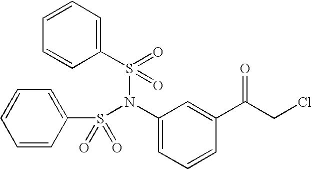

2.1 ml (26 mmol) sulphuryl chloride over a period of 20 minutes are added dropwise at 0° C. with vigorous stirring to 3.6 g (8.66 mmol) N-(3-acetyl-phenyl)-dibenzenesulphonamide in 70 ml dichloromethane and 2.11 ml (52 mmol) methanol. The reaction mixture is refluxed for 2.5 hours and then stirred for 18 hours at ambient temperature. Then the reaction solution is washed with water, saturated aqueous sodium hydrogen carbonate solution and saturated aqueous sodium chloride solution. The organic phase is separated off, dried on magnesium sulphate and concentrated by evaporation in the rotary evaporator. The residue is recrystallised from toluene to obtain a colourless solid.

Yield: 2.55 g (65 % of theory)

C₂₀H₁₆ClNO₅S₂ (449.93)

MS [ESI (M+NH₄)⁺]=459, 457

R_{f}=0.56 [silica gel, toluene/ethyl acetate (9/1)]

### Step 3: N—[(R)-3-oxiranyl-phenyl]-dibenzenesulphonamide

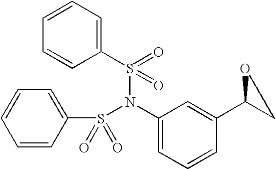

7.84 g (24.4 mmol) (−)-B-chloro-diisopinocampheylboran dissolved in 15 ml of tetrahydrofuran are added dropwise at −30° C. over a period of 60 minutes to a solution of 5.00 g (11.1 mmol) N-[3-(2-chloro-acetyl)-phenyl]-dibenzenesulphonamide in 70 ml of tetrahydrofuran. After one hour another 2.00 g (6.24 mmol) (−)-B-chloro-diisopinocampheylboran dissolved in 5 ml of tetrahydrofuran are added dropwise at −30° C. The mixture is stirred for 14 hours at this temperature and the reaction solution is then poured into a mixture of ice water and saturated sodium hydrogen carbonate solution. The mixture is extracted with ethyl acetate, the combined organic phases are washed and dried on magnesium sulphate. Then the mixture is evaporated to dryness. The residue is chromatographed on silica gel (toluene/ethyl acetate=97.5:2.5→90:10). The intermediate product is triturated with diisopropylether, suction filtered and dried. The solid is dissolved in 30 ml DMF and combined with 8.33 ml of 4 N lithium hydroxide solution at −5° C. with stirring within 15 minutes. Meanwhile 3 ml DMF and 2 ml of water are added to improve the stirrability. After 25 minutes the reaction mixture is acidified with glacial acetic acid at −5° C. and diluted with water. The solid thus precipitated is suction filtered, washed several times with ice water and dried. (The product may be obtained in racemic form by reacting N-[3-(2-chloro-acetyl)-phenyl]-dibenzenesulphonamide with borane-tetrahydrofuran complex (1 M in tetrahydrofuran) and then with 4 M lithium hydroxide.)

Yield: 3.65 g (79% of theory)

C₂₀H₁₇NO₅S₂ (415.49)

MS [ESI (M+NH₄)⁺]=433

R_{f}=0.47 [silica gel, toluene/ethyl acetate (9/1)]

Preparation of the End Compounds

### EXAMPLE 1

### N-{3-[2-(3-benzimidazol-1-yl-1,1-dimethyl-propylamino)-1-hydroxy-ethyl]-phenyl}-benzenesulphonamide

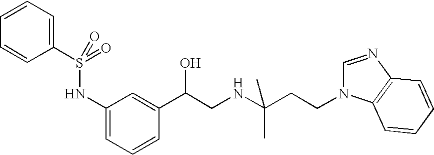

A solution of 0.300 g (0.900 mmol) N-[3-(2-ethoxy-2-hydroxyacetyl)-phenyl]-benzenesulphonamide and 0.215 mg (0.900 mmol) 3-benzimidazol-1-yl-1,1-dimethyl-propylamine hydrochloride in 10 mL ethanol is stirred for 16 hours at 80° C. The reaction mixture is left to come up to ambient temperature, and 0.135 g (3.60 mmol) sodium borohydride are added batchwise. The mixture is stirred for a further 2 hours and then combined with 0.5 mL water. The precipitate is suction filtered and washed with diethyl ether. The residue is triturated with diethyl ether, to obtain 0.170 g (0.355, 40%) N-{3-[2-(3-benzimidazol-1-yl-1,1-dimethyl-propylamino)-1-hydroxy-ethyl]-phenyl}-benzenesulphonamide.

R_{f}=0.10 [silica gel, dichloromethane/methanol/ammonia (95/5/0.1)]

MS [ESI (M+H)⁺]=479

### EXAMPLE 2

### N-(3-{2-[3-(5,6-dichloro-benzimidazol-1-yl)-1,1-dimethyl-propylamino]-1-hydroxy-ethyl}-phenyl)-benzenesulphonamide trifluoroacetate

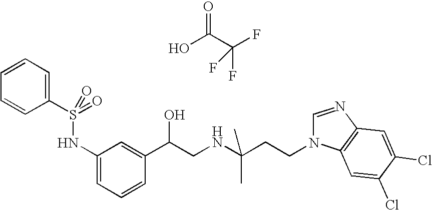

0.067 g (0.200 mmol) N-[3-(2-ethoxy-2-hydroxy-acetyl)-phenyl]-benzenesulphonamide and 0.048 g (0.139 mmol) 3-(5,6-dichloro-benzimidazol-1-yl)-1,1-dimethyl-propylamine dihydrochloride are dissolved in 2 mL ethanol and the pH of the reaction mixture is adjusted to 8-9 with triethylamine. The reaction mixture is refluxed for 16 hours, then cooled to 0° C. and combined with 0.023 g (0.600 mmol) sodium borohydride. The mixture is stirred for a further 2 hours at ambient temperature and then the pH of the reaction mixture is adjusted to <2 with trifluoroacetic acid. Purification by reversed-phase flash column chromatography {Varian Microsorb C18-reverse-phase [acetonitrile (0.1% trifluoroacetic acid)/water (0.13% trifluoroacetic acid)=10:90→100:0]} yielded 0.045 g (0.068 mmol, 34 %) N-(3-{2-[3-(5,6-dichloro-benzimidazol-1-yl)-1,1-dimethyl-propylamino]-1-hydroxy-ethyl}-phenyl)-benzenesulphonamide trifluoroacetate.

R_{f}=0.44 [silica gel, dichloromethane/methanol/ammonia (90/10/0.1)]

MS [ESI (M+H)⁺]=547/549 (Cl)

### EXAMPLE 3

### N-{3-[2-(1,1-dimethyl-3-naphtho[2.3-d]imidazol-1-yl-propylamino)-1-hydroxy-ethyl]-phenyl}-benzenesulphonamide trifluoroacetate

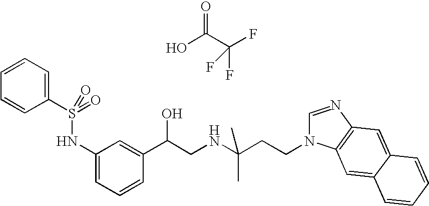

0.300 g (0.895 mmol) N-[3-(2-ethoxy-2-hydroxy-acetyl)-phenyl]-benzenesulphonamide and 0.227 g (0.895 mmol) 1,1-dimethyl-3-naphtho[2,3-d]imidazol-1-yl-propylamine are refluxed for 16 hours in 10 mL ethanol. The reaction mixture is then cooled to 0° C. and combined with 0.135 g (3.58 mmol) sodium borohydride. The mixture is stirred for a further 2 hours at ambient temperature, 0.5 mL water is added and then the pH of the reaction mixture is adjusted to <2 with trifluoroacetic acid. Purification by reversed-phase flash column chromatography {Varian Microsorb C18-reverse-phase [acetonitrile (0.1% trifluoroacetic acid)/water (0.13% trifluoroacetic acid)=10:90→100:0]} yielded 0.220 g (0.342 mmol, 38 %) N-{3-[2-(1,1-dimethyl-3-naphtho[2,3-d]imidazol-1-yl-propylamino)-1-hydroxy-ethyl]-phenyl}-benzenesulphonamide trifluoroacetate.

R_{f}=0.21 [silica gel, dichloromethane/methanol/ammonia (95/5/0.1)]

MS [ESI (M+H)⁺]=529

### EXAMPLE 4

### N-(3-{2-[3-(6-amino-benzimidazol-1-yl)-1,1-dimethyl-propylamino]-1-hydroxy-ethyl}-phenyl)-benzenesulphonamide

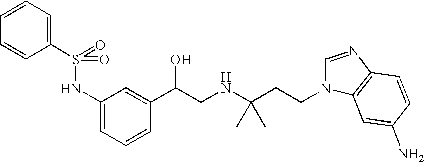

### Step 1: 1,1-dimethyl-3-(6-nitro-benzimidazol-1-yl)-propylamine

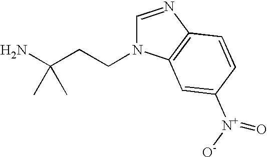

0.648 g (27.0 mmol) sodium hydride (95%) are added at 10° C. to 3.90 g (24.0 mmol) 6-nitro-1H-benzimidazole in 5 mL 1,3-dimethyl-3.4.5,6-tetrahydro-2(1H)-pyrimidone and stirred for 1 hour at 10° C. The reaction mixture is then combined with 7.522 g (27.0 mmol) 3-chloro-1,1-dimethyl-propyl)-(2,6-dichloro-benzylidene)-amine and 0.887 g (2.40 mmol) tetrabutylammonium iodide and stirred for 72 hours at ambient temperature and for 5 hours at 100° C. The reaction mixture is poured into ice water and extracted with ethyl acetate. The combined organic phases are washed with water, dried on magnesium sulphate and concentrated by evaporation in the rotary evaporator.

The residue is dissolved in 10 hydrochloric acid (3.5 M) and refluxed for 1 hour. The reaction mixture is neutralised with NaOH and extracted with ethyl acetate. The combined organic phases are dried on magnesium sulphate and concentrated by evaporation in the rotary evaporator. The residue is purified by flash column chromatography [silica gel, dichloromethane/methanol/ammonia (100/0/0→80/20/1)]. 1.91 g (80.0 mmol, 32%) 1,1-dimethyl-3-(6-nitro-benzimidazol-1-yl)-propylamine are obtained.

R_{f}=0.25 [silica gel, dichloromethane/methanol/ammonia (9/1/0.1)]

MS [ESI (M−H)⁻]=247

### Step 2: N-(3-{2-[1,1-dimethyl-3-(6-nitro-benzimidazol-1-yl)-propylamino]-1-hydroxy-ethyl}-phenyl)-benzenesulphonamide

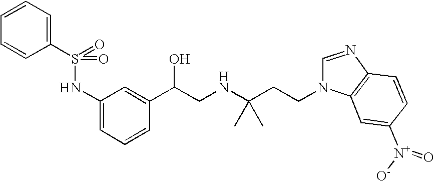

A solution of 0.335 g (1.00 mmol) N-[3-(2-ethoxy-2-hydroxyacetyl)-phenyl]-benzenesulphonamide and 0.248 mg (1.00 mmol) 1,1-dimethyl-3-(6-nitro-benzimidazol-1-yl)-propylamine in 10 mL ethanol is stirred for 15 hours at 80° C. The reaction mixture is cooled to 0° C. and 0.113 g (3.00 mmol) sodium borohydride are added batchwise. The mixture is stirred for a further 6 hours at ambient temperature, poured into 20 mL saturated, aqueous potassium carbonate solution and then extracted with ethyl acetate. The combined organic phases are washed with saturated aqueous sodium chloride solution, dried on sodium sulphate and concentrated by evaporation in the rotary evaporator. The residue is purified by flash column chromatography [silica gel, dichloromethane/methanol/(100/0→75/25)], to obtain 0.310 g (0.592 mmol, 59%) N-(3-{2-[1,1-dimethyl-3-(5-nitro-benzimidazol-1-yl)propylamino]-1-hydroxy-ethyl}-phenyl)-benzenesulphonamide.

R_{f}=0.29 [silica gel, dichloromethane/methanol (90/10)]

MS [ESI (M+H)⁺]=524

### Step 3: N-(3-{2-[3-(6-amino-benzimidazol-1-yl)-1,1-dimethyl-propylamino]-1-hydroxy-ethyl}-phenyl)-benzenesulphonamide

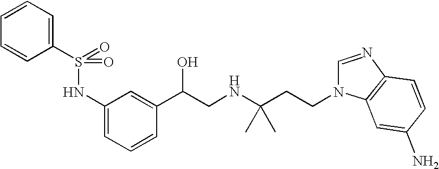

0.200 g (0.382 mmol) N-(3-{2-[1,1-dimethyl-3-(6-nitro-benzimidazol-1-yl)-propyl-amino]-1-hydroxy-ethyl}-phenyl)-benzenesulphonamide, 0.050 g palladium on charcoal in 10 mL methanol are shakenat ambient temperature in an autoclave at 3 bar hydrogen atmosphere for 6 hours. The reaction mixture is filtered and the filtrate is concentrated by evaporation in the rotary evaporator. 0.185 g (0.375 mmol, 98%) N-(3-{2-[3-(6-amino-benzimidazol-1-yl)-1,1-dimethyl-propylamino]-1-hydroxy-ethyl}-phenyl)-benzenesulphonamide are obtained.

R_{f}=0.20 [silica gel, dichloromethane/methanol/ammonia (90/9/1)]

MS [ESI (M+H)⁺]=494

### EXAMPLE 5

### 1-{3-[2-(3-benzenesulphonylamino-phenyl)-2-hydroxy-ethylamino]-3-methyl-butyl}-1H-benzimidazole-4-carboxylic acid trifluoroacetate

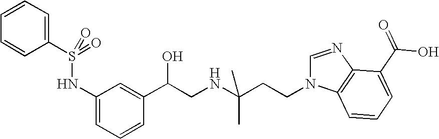

### Step 1: ethyl 1H-benzimidazole-4-carboxylate

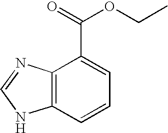

2.50 g (15.4 mmol) 1H-benzimidazole-4-carboxylic acid in 100 mL ethanol are combined with 50 mL saturated ethanolic hydrochloric acid solution and refluxed for 15 hours. The reaction mixture is poured into saturated, aqueous potassium carbonate solution and extracted with ethyl acetate. The combined organic phases are dried on sodium sulphate and evaporated down to 50 mL using the rotary evaporator and filtered. 1.80 g (9.46 mmol, 61%) ethyl 1H-benzimidazole-4-carboxylate are obtained as a colourless solid.

R_{f}=0.92 [silica gel, dichloromethane/methanol (80/20)]

MS [ESI (M+H)⁺]]=191

### Step 2: ethyl 1-(3-tert-butoxycarbonylamino-3-methyl-butyl)-1H-benzimidazole-4-carboxylate

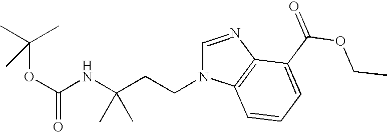

0.502 g (12.6 mmol) sodium hydride (60% in mineral oil) are added batchwise at 5° C. to 3.09 g (13.9 mmol) ethyl 1H-benzimidazole-4-carboxylate in 15 mL dimethylformamide and stirred for 30 minutes at ambient temperature. The reaction mixture is then combined with 1.75 g (9.20 mmol) tert-butyl (3-chloro-1,1-dimethyl-propyl)-carbamate and 0.310 g (0.840 mmol) tetraabutylammonium iodide and stirred for 24 hours at 60° C. The reaction mixture is poured into ice water and the aqueous phase is extracted with ethyl acetate. The combined organic phases are dried on sodium sulphate and concentrated by evaporation in the rotary evaporator. The residue is purified by reversed-phase flash column chromatography {Varian Microsorb C18-reverse-phase [acetonitrile (0.1% trifluoroacetic acid)/water (0.13% trifluoroacetic acid)=10:90→100:0]}, thus obtaining 0.850 g (2.26 mmol, 16%) ethyl 1-(3-tert-butoxycarbonylamino-3-methyl-butyl)-1H-benzim idazole-4-carboxylate.

R_{f}=0.48 [silica gel, dichloromethane/methanol/ammonia (95/5/0.1)]

MS [ESI (M+H)⁺]=376

### Step 3: ethyl 1-(3-amino-3-methyl-butyl)-1H-benzimidazole-4-carboxylate

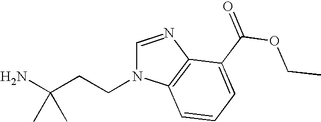

0.850 g (2.26 mmol) ethyl 1-(3-tert-butoxycarbonylamino-3-methyl-butyl)-1H-benzimidazole-4-carboxylate are dissolved in 3 mL dichloromethane and combined with 3 mL trifluoroacetic acid. The reaction mixture is stirred for 4 hours at 40° C. and then concentrated by evaporation in the rotary evaporator. The residue is purified by reversed-phase flash column chromatography {Varian Microsorb C18-reverse-phase [acetonitrile (0.1% trifluoroacetic acid)/water (0.13% trifluoroacetic acid)=10:90→100:0]}, thus obtaining 0.620 g (2.25 mmol, 99%) ethyl 1-(3-amino-3-methyl-butyl)-1H-benzimidazole-4-carboxylate.

R_{f}=0.51 [silica gel, dichloromethane/methanol/ammonia (90/10/0.1)]

MS [ESI (M+H)⁺]=276

### Step 4: ethyl 1-{3-[2-(3-benzenesulphonylamino-phenyl)-2-hydroxy-ethylamino]-3-methyl-butyl}-1H-benzimidazole-4-carboxylate

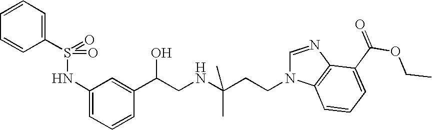

0.300 g (0.895 mmol) N-[3-(2-ethoxy-2-hydroxy-acetyl)-phenyl]-benzenesulphonamide and 0.246 g (0.895 mmol) ethyl 1-(3-amino-3-methyl-butyl)-1H-benzimidazole-4-carboxylate are dissolved in 10 mL ethanol and the pH value of the reaction mixture is adjusted to 8-9 with triethylamine. The reaction mixture is refluxed for 16 hours, then cooled to ambient temperature and combined with 0.135 g (0.358 mmol) sodium borohydride. The mixture is stirred for a further 2 hours at ambient temperature and then the pH of the reaction mixture is adjusted to <2 with trifluoroacetic acid. Purification by reversed-phase flash column chromatography {Varian Microsorb C18-reverse-phase [acetonitrile (0.1% trifluoroacetic acid)/water (0.13% trifluoroacetic acid)=10:90→100:0]} yielded 0.120 g (0.218 mmol, 24%) ethyl 1-{3-[2-(3-benzenesuphonylamino-phenyl)-2-hydroxy-ethylamino]-3-methyl-butyl}-1H-benzimidazole-4-carboxylate.

R_{f}=0.37 [silica gel, dichloromethane/methanol/ammonia (9/1/0.1)]

MS [ESI (M+H)⁺]=551

### Step 5: 1-{3-[2-(3-benzenesulphonylamino-phenyl)-2-hydroxy-ethylamino]-3-methyl-butyl}-1H-benzimidazole-4-carboxylic acid trifluoroacetate

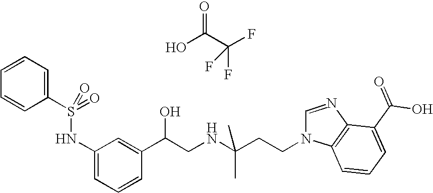

0.100 g (0.182 mmol) ethyl 1-{3-[2-(3-benzenesulphonylamino-phenyl)-2-hydroxy-ethylamino]-3-methyl-butyl}-1H-benzimidazole-4-carboxylate are dissolved in 5 mL ethanol and combined with 5 mL lithium hydroxide solution (2M in water). The reaction mixture is stirred for 3 hours at ambient temperature and then adjusted to a pH <2 with trifluoroacetic acid. Purification by reversed-phase flash column chromatography {Varian Microsorb C18-reverse-phase [acetonitrile (0.1% trifluoroacetic acid)/water (0.13% trifluoroacetic acid)=10:90→100:0]} yielded 0.070 g (0.110 mmol, 61%) -{3-[2-(3-benzenesulphonylamino-phenyl)-2-hydroxy-ethylamino]-3-methyl-butyl}-1H-benzimidazole-4-carboxylic acid trifluoroacetate.

R_{f}=0.26 [silica gel, dichloromethane/methanol/ammonia (80/20/0.1)]

MS [ESI (M+H)⁺]=523

### Example 6

### Ethyl (R)-1-{3-[2-(3-benzenesulphonylamino-phenyl)-2-hydroxy-ethylamino]-3-methyl-butyl}-1H-benzimidazole-5-carboxylate

### Step 1: ethyl 4-fluoro-3-nitro-benzoate

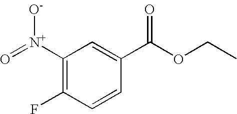

5.00 g (27.0 mmol) 4-fluoro-3-nitro-benzoic acid are refluxed for 15 hours in 50 mL saturated, ethanolic hydrochloric acid solution (1.25 M). The reaction mixture is evaporated down using the rotary evaporator and purified by flash column chromatography [silica gel, petroleum ether/ethyl acetate (4/1)]. 5.48 g (25.7 mmol, 95%) ethyl 4-fluoro-3-nitro-benzoate are obtained as a colourless solid.

R_{f}=0.48 [silica gel, petroleum ether/ethyl acetate (4/1)]

MS [ESI (M+H)⁺]=214

### Step 2: ethyl 4-(3-methyl-3-nitro-butylamino)-3-nitro-benzoate

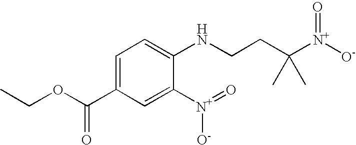

5.80 g (27.2 mmol) 2,6-dichloro-3-nitropyridine are added at ambient temperature to 4.59 g (27.2 mmol) 3-methyl-3-nitro-butylamin-hydrochloride and 10.2 mL (59.9 mmol) diisopropylethylamine in 150 mL acetonitrile. The reaction mixture is stirred for 7 days at ambient temperature and then concentrated by evaporation in the rotary evaporator. The residue is purified by flash column chromatography [silica gel, petroleum ether/ethyl acetate (4/1)]. 8.56 g (26.3 mmol, 97%) ethyl 4-(3-methyl-3-nitro-butylamino)-3-nitro-benzoate are obtained.

R_{f}=0.16 [silica gel, petroleum ether/ethyl acetate (4/1)]

MS [ESI (M+H)⁺]=326

### Step 3: ethyl 1-(3-amino-3-methyl-butyl)-1H-benzimidazole-5-carboxylate

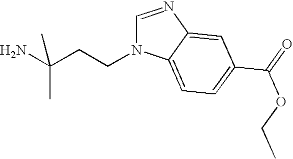

6.4 g (19.7 mmol) ethyl 4-(3-methyl-3-nitro-butylamino)-3-nitro-benzoate, 1.00 g palladium on charcoal and 50 mL methanol in 50 mL tetrahydrofuran are shaken at ambient temperature in an autoclave at 3 bar hydrogen atmosphere for 24 hours. The reaction mixture is filtered and the filtrate is concentrated by evaporation in the rotary evaporator.

The residue is stirred for 3 hours at 100° C. in 150 mL formic acid. The reaction mixture is evaporated down using the rotary evaporator and the residue is purified by flash column chromatography [silica gel, petroleum ether/ethyl acetate (4/1)]. 5.29 g (19.2 mmol, quantitative %) ethyl 1-(3-amino-3-methyl-butyl)-1H-benzimidazole-5-carboxylate are obtained.

R_{f}=0.36 [silica gel, dichloromethane/methanol/ammonia (60/10/0.1)]

MS [ESI (M+H)⁺]=276

### Step 4: ethyl (R)-1-{3-[2-(3-benzenesulphonylamino-phenyl)-2-hydroxy-ethylamino]-3-methyl-butyl}-1H-benzimidazole-5-carboxylate

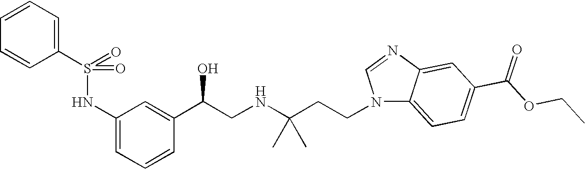

2.85 g (10.4 mmol) ethyl 1-(3-amino-3-methyl-butyl)-1H-benzimidazole-5-carboxylate and 2.40 g (8.72 mmol) (R)—N-(3-oxiranyl-phenyl)-benzenesulphonamide are stirred for 45 minutes at 100° C. The reaction mixture is purified by reversed-phase flash column chromatography {Varian Microsorb C18-reverse-phase [acetonitrile (0.1% trifluoroacetic acid)/water (0.13% trifluoroacetic acid)=10:90→100:0]}, thus obtaining 0.900 g (1.63 mmol, 19%) ethyl (R)-1-{3-[2-(3-benzenesulphonylamino-phenyl)-2-hydroxy-ethylamino]-3-methyl-butyl}-1H-benzimidazole-5-carboxylate.

R_{f}=0.41 [silica gel, dichloromethane/methanol (9/1)]

MS [ESI (M+H)⁺]=551

### EXAMPLE 7

(R)-1-{3-[2-(3-benzenesulphonylamino-phenyl)-2-hydroxy-ethylamino]-3-methyl-butyl}-1H-benzimidazole-5-carboxylic acid-trifluoroacetate

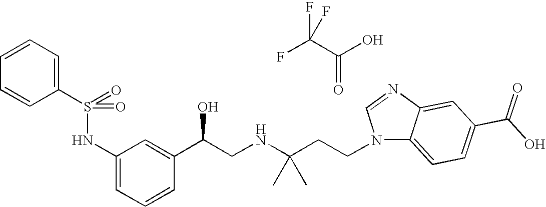

0.300 g (0.545 mmol) ethyl (R)-1-{3-[2-(3-benzenesulphonylamino-phenyl)-2-hydroxy-ethylamino]-3-methyl-butyl}-1H-benzimidazole-5-carboxylate are dissolved in 1.5 mL ethanol and combined with 2 mL lithium hydroxide solution (2M in water).

The reaction mixture is stirred for 18 hours at ambient temperature and then adjusted with trifluoroacetic acid to a pH <2. Purification by reversed-phase flash column chromatography {Varian Microsorb C18-reverse-phase [acetonitrile (0.1% trifluoroacetic acid)/water (0.13% trifluoroacetic acid)=10:90→100:0]} yielded 0.300 g (0.471 mmol, 87%) (R)-1-{3-[2-(3-benzenesulphonylamino-phenyl)-2-hydroxy-ethylamino]-3-methyl-butyl}-1H-benzimidazole-5-carboxylic acid trifluoroacetate.

R_{f}=0.33 [silica gel, acetonitrile/water/glacial acetic acid (35/65/0.2)]

MS [ESI (M+H)⁺]=521

### EXAMPLE 8

### Ethyl 1-{3-[2-(3-benzenesulphonylamino-phenyl)-2-hydroxy-ethylamino]-3-methyl-butyl}-1H-benzimidazole-6-carboxylate

### Step 1: ethyl 1H-benzimidazole-5-carboxylate

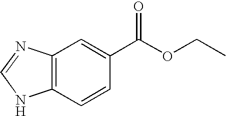

Patent Kyowa Hakko Kogyo Co., Ltd., U.S. Pat. No. 5,053,408

### Step 2 ethyl 1-(3-tert-butoxycarbonylamino-3-methyl-butyl)-1H-benzimidazole-6-carboxylate

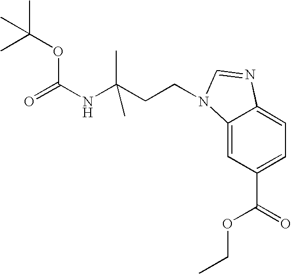

1.85 g (46.0 mmol) sodium hydride (60% in mineral oil) are added batchwise at 5° C. to 8.00 g (42.0 mmol) ethyl 1H-benzimidazole-5-carboxylate in 50 mL 1,3-dimethyl-3,4,5,6-tetrahydro-2(1H)-pyrimidone (DMPU) and stirred for 30 minutes at ambient temperature. The reaction mixture is then combined with 9.312 g (42.0 mmol) tert-butyl (3-chloro-1,1-dimethyl-propyl)-carbamate (dissolved in 30 mL DMPU) and 1.55 g (4.20 mmol) tetrabutylammonium iodide and stirred for 18 hours at 60° C. The reaction mixture is poured into ice water and the aqueous phase is extracted with ethyl acetate. The combined organic phases are washed with water and saturated aqueous sodium chloride solution, dried on sodium sulphate and concentrated by evaporation in the rotary evaporator. The residue is purified by flash column chromatography [silica gel, dichloromethane/methanol (90/10)], thus obtaining 11.2 g (30.0 mmol, 71%) ethyl 3-(3-tert-butoxycarbonylamino-3-methyl-butyl)-1H-benzimidazole-6-carboxylate (1:1 mixture of regioisomers with ethyl 1-(3-tert-butoxycarbonylamino-3-methyl-butyl)-1H-benzimidazole-5-carboxylate).

R_{f}=0.60 [silica gel, dichloromethane/methanol (90/10)]

MS [ESI (M+H)⁺]=376

### Step 3: ethyl 1-(3-amino-3-methyl-butyl)-1H-benzimidazole-6-carboxylate

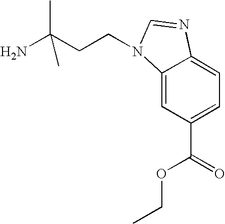

11.2 g (30.0 mmol) ethyl 1-(3-tert-butoxycarbonylamino-3-methyl-butyl)-1H-benzimidazole-6-carboxylate (1:1 mixture of regioisomers with ethyl 1-(3-tert-butoxy-carbonylamino-3-methyl-butyl)-1H-benzimidazole-5-carboxylate) are dissolved in 100 mL dichloromethane and at 0° C. combined with 30 mL trifluoroacetic acid. The reaction mixture is stirred for 18 hours at ambient temperature and then concentrated by evaporation in the rotary evaporator. The residue is taken up in ethyl acetate and combined with sodium hydroxide solution (1M) until the aqueous phase has a pH of 8-9. The phases are separated and the aqueous phase is extracted with dichloromethane. The combined organic phases are dried on sodium sulphate and concentrated by evaporation in the rotary evaporator. 7.60 g (28.0 mmol, 92%) ethyl 1-(3-amino-3-methyl-butyl)-1H-benzimidazole-6-carboxylate (1:1 mixture of regioisomers with ethyl 1-(3-amino-3-methyl-butyl)-1H-benzimidazole-5-carboxylate) are obtained.

R_{f}=0.36 [silica gel, dichloromethane/methanol/ammonia (60/10/0.1)]

MS [ESI (M+H)⁺]=276

### Step 4: ethyl 1-{3-[2-(3-benzenesulphonylamino-phenyl)-2-hydroxy-ethylamino]-3-methyl-butyl}-1 H-benzimidazole-6-carboxylate

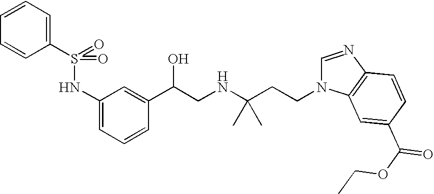

0.731 g (2.18 mmol) N-[3-(2-ethoxy-2-hydroxy-acetyl)-phenyl]-benzenesulphonamide and 0.600 g (2.18 mmol) ethyl 1-(3-amino-3-methyl-butyl)-1H-benzimidazole-6-carboxylate are refluxed for 72 hours in 20 mL ethanol. The reaction mixture is cooled to 0° C. and combined with 0.082 g (2.18 mmol) sodium borohydride. The mixture is stirred for another 4 hours at ambient temperature and then the pH of the reaction mixture is adjusted to <2 with hydrochloric acid (1M). The reaction mixture is evaporated down using the rotary evaporator, the residue in water and extracted with ethyl acetate. The combined organic phases are discarded, the aqueous phase is adjusted to a pH of 8-9 with concentrated aqueous ammonia solution and extracted with dichloromethane. The combined organic phases are dried on sodium sulphate and concentrated by evaporation in the rotary evaporator. The residue is purified by flash column chromatography [silica gel, dichloromethane/methanol/ammonia (190/9/1)], thus obtaining 0.020 g (0.031 mmol, 1.4%) ethyl 1-{3-[2-(3-benzenesulphonylamino-phenyl)-2-hydroxy-ethylamino]-3-methyl-butyl}-1H-benzimidazole-5-carboxylate.

R_{f}=0.33 [silica gel, dichloromethane/methanol/ammonia (90/9/1)]

MS [ESI (M+H)⁺]=551

### EXAMPLE 9

### Ethyl (R)-1-{3-[2-(3-benzenesulphonylamino-phenyl)-2-hydroxy-ethylamino]-3-methyl-butyl}-1H-benzimidazole-6-carboxylate

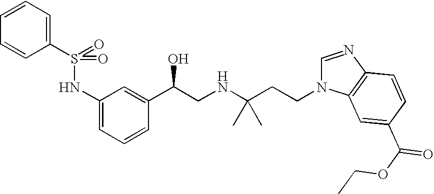

Ethyl 1-(3-amino-3-methyl-butyl)-1H-benzimidazole-6-carboxylate is prepared in 4 steps from ethyl 3-fluoro-4-nitro-benzoate (analogously to Steps 1-4 in Example 6). 2.40 g (8.72 mmol) ethyl 1-(3-amino-3-methyl-butyl)-1H-benzimidazole-6-carboxylate and 2.88 g (10.5 mmol) (R)—N-(3-oxiranyl-phenyl)-benzenesulphonamide are stirred for 45 minutes at 100° C. The reaction mixture is purified by reversed-phase flash column chromatography {Varian Microsorb C18-reverse-phase [acetonitrile (0.1% trifluoroacetic acid)/water (0.13% trifluoroacetic acid)=10:90→100:0]}, thus obtaining 1.15 g (2.09 mmol, 24%) ethyl (R)-1-{3-[2-(3-benzenesulphonylamino-phenyl)-2-hydroxy-ethylamino]-3-methyl-butyl}-1H-benzimidazole-6-carboxylate.

R_{f}=0.42 [silica gel, dichloromethane/methanol (9/1)]

MS [ESI (M+H)⁺]=551

### EXAMPLE 10

### (R)-1-{3-[2-(3-benzenesulphonylamino-phenyl)-2-hydroxy-ethylamino]-3-methyl-butyl}-1H-benzimidazole-6-carboxylic acid hydrotrifluoroacetate

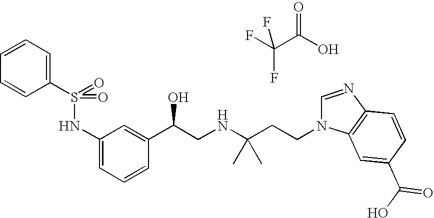

0.300 g (0.545 mmol) ethyl (R)-1-{3-[2-(3-benzenesulphonylamino-phenyl)-2-hydroxy-ethylamino]-3-methyl-butyl}-1H-benzimidazole-6-carboxylate are dissolved in 1.5 mL ethanol and combined with 2 mL lithium hydroxide solution (2M in water). The reaction mixture is stirred for 18 hours at ambient temperature and then adjusted with trifluoroacetic acid to a pH <2. Purification by reversed-phase flash column chromatography {Varian Microsorb C18-reverse-phase [acetonitrile (0.1% trifluoroacetic acid)/water (0.13% trifluoroacetic acid)=10:90→100:0]} yielded 0.330 g (0.518 mmol, 95%) (R)-1-{3-[2-(3-benzenesulphonylamino-phenyl)-2-hydroxy-ethylamino]-3-methyl-butyl}-1H-benzimidazole-6-carboxylic acid trifluoroacetate.

R_{f}=0.35 [silica gel, acetonitrile/water/glacial acetic acid (35/65/0.2)]

MS [ESI (M+H)⁺]=523

### EXAMPLE 11

### N-(3-{2-[1,1-dimethyl-3-(2-methyl-benzimidazol-1-yl)-propylamino]-1-hydroxy-ethyl}-phenyl)-benzenesulphonamide

### Step 1: 1,1-dimethyl-3-(2-methyl-benzimidazol-1-yl)-propylamine dihydrochloride

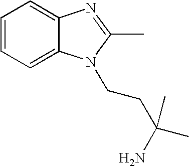

2.20 g (55.0 mmol) sodium hydride (60% in mineral oil) are added at 10° C. to 6.60 g (49.9 mmol) 2-methyl-1H-benzimidazole in 100 mL dimethylformamide and stirred for 1 hour at 10° C. The reaction mixture is then combined with 14.0 g (50.3 mmol) 3-chloro-1,1-dimethyl-propyl)-(2,6-dichloro-benzylidene)-amine (dissolved in 50 mL dimethylformamide) and 0.830 g (5.00 mmol) potassium iodide and stirred for 24 hours at 85° C. The reaction mixture is poured into 600 mL ice water, combined with 30 mL concentrated hydrochloric acid and extracted with ethyl acetate. The organic phase is discarded, the aqueous phase is adjusted to pH >9 with potassium carbonate and extracted with dichloromethane. The combined organic phases are dried on magnesium sulphate and concentrated by evaporation in the rotary evaporator.

The residue is dissolved in ethyl acetate and combined with hydrogen chloride (saturated solution in ethyl acetate). The reaction mixture is filtered and the solid obtained is washed with ethyl acetate and dried. 5.20 g (23.9 mmol, 48%) 1,1-dimethyl-3-(2-methyl-benzimidazol-1-yl)-propylamine dihydrochloride are obtained.

m.p. =318-320° C.

MS [ESI (M+H)⁺]=218

### Step 2: N-(3-{2-[1,1-dimethyl-3-(2-methyl-benzimidazol-1-yl)-propylamino]-1-hydroxy-ethyl}-phenyl)-benzenesulphonamide hydrotrifluoroacetate

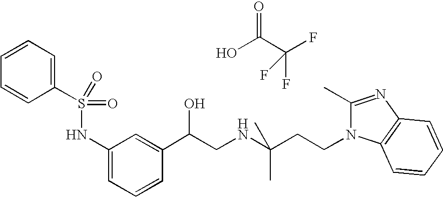

0.300 g (0.895 mmol) N-[3-(2-ethoxy-2-hydroxy-acetyl)-phenyl]-benzenesulphonamide and 0.260 g (0.895 mmol) 1,1-dimethyl-3-(2-methyl-benzimidazol-1-yl)-propylamine dihydrochloride are refluxed for 16 hours in 10 mL ethanol. The reaction mixture is cooled to ambient temperature and combined with 0.135 g (3.58 mmol) sodium borohydride. The mixture is stirred for a further 2 hours at ambient temperature and then the pH of the reaction mixture is adjusted to <2 with trifluoroacetic acid. Purification by reversed-phase flash column chromatography {Varian Microsorb C18-reverse-phase [acetonitrile (0.1% trifluoroacetic acid)/water (0.13% trifluoroacetic acid)=10:90→100:0]} yielded 0.120 g (0.198 mmol, 22%) N-(3-{2-[1,1-dimethyl-3-(2-methyl-benzimidazol-1-yl)-propylamino]-1-hydroxy-ethyl}-phenyl)-benzenesulphonamide.

R_{f}=0.09 [silica gel, dichloromethane/methanol/ammonia (95/5/0.1)]

MS [ESI (M+H)⁺]=493

### EXAMPLE 12

### N-(3-{2-[1,1-dimethyl-3-(2-oxo-2,3-dihydro-benzimidazol-1-yl)-propylamino]-1-hydroxy-ethyl}-phenyl)-benzenesulphonamide

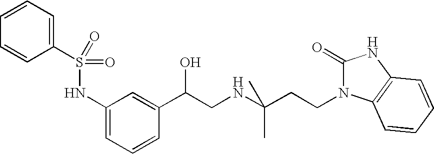

0.300 g (0.895 mmol) N-[3-(2-ethoxy-2-hydroxy-acetyl)-phenyl]-benzenesulphonamide and 0.260 g (0.895 mmol) 1-(3-amino-3-methyl-butyl)-1,3-dihydro-benzimidazole-2-on trifluoroacetate are dissolved in 10 mL ethanol and the pH of the reaction mixture is adjusted to 8-9 with triethylamine. The reaction mixture is refluxed for 16 hours, then cooled to ambient temperature and combined with 0.135 g (0.358 mmol) sodium borohydride. The mixture is stirred for a further 2 hours at ambient temperature and then filtered. The solid obtained is washed with diethyl ether and dried. 0.230 g (0.465 mmol, 52%) N-(3-{2-[1,1-dimethyl-3-(2-oxo-2,3-dihydro-benzimidazol-1-yl)-propylamino]-1-hydroxy-ethyl}-phenyl)-benzenesulphonamide are obtained.

R_{f}=0.46 [silica gel, dichloromethane/methanol/ammonia (90/10/0.1)]
MS [ESI (M+H)⁺]=495

### EXAMPLE 13

### methyl 1-{3-[2-(3-benzenesulphonylamino-phenyl)-2-hydroxy-ethylamino]-3-methyl-butyl}-2-oxo-2,3-dihydro-1H-benzimidazole-5-carboxylate

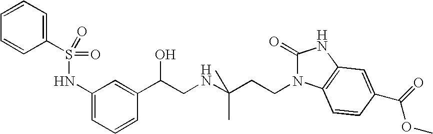

### Step 1: tert-butyl (3-hydroxy-1,1-dimethyl-propyl)-carbamate

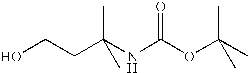

88.2 g (404 mmol) di-tert.-butyldicarbonate are added batchwise at ambient temperature to a solution of 41.3 g (400 mmol) 3-amino-3-methyl-butanol in 250 mL ethyl acetate. The reaction mixture is stirred for 18 hours at RT and then concentrated by evaporation in the rotary evaporator. 81.3 g (400 mmol, quantitative) tert-butyl (3-amino-1,1-dimethyl-propyl)-carbamate are obtained.

R_{f}=0.48 [silica gel, petroleum ether/ethyl acetate (1/1)]

MS [ESI (M+H)⁺]=204

### Step 2: tert-butyl (3-amino-1,1-dimethyl-propyl)-carbamate

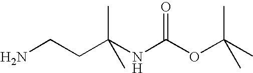

38.6 g (242.7 mmol) sulphur trioxide-pyridine complex are added at ambient temperature to 31.9 g (157 mmol) tert-butyl (3-amino-1,1-dimethyl-propyl)-carbamate and 63.3 mL (455 mmol) triethylamine in 400 mL dimethylsulphoxide. The reaction mixture is stirred for 72 hours at ambient temperature and then combined with 200 mL toluene and 300 mL water. The phases are separated and the aqueous phase is extracted with toluene. The combined organic phases are washed successively with aqueous potassium hydrogen sulphate solution (0.5 M), aqueous sodium hydrogen carbonate solution (10% ish) and water, dried on sodium sulphate and concentrated by evaporation in the rotary evaporator.

The residue is dissolved in 700 mL saturated, ethanolic ammonia and at ambient temperature combined with 3.52 g Raney nickel and shaken for 24 hours at ambient temperature in an autoclave at 3 bar hydrogen atmosphere. The reaction mixture is filtered and the filtrate concentrated by evaporation in the rotary evaporator. 22.7 g (112 mmol, 28%) tert-butyl (3-amino-1,1-dimethyl-propyl)-carbamate are obtained.

R_{f}=0.15 [silica gel, dichloromethane/methanol (90/10)]

MS [ESI (M+H)⁺]=203

### Step 3: 4-(3-tert-butoxycarbonylamino-3-methyl-butylamino)-3-nitro-benzoic acid

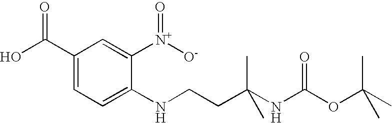

6.00 g (32.4 mmol) 4-fluoro-3-nitro-benzoic acid, 6.83 g (33.8 mmol) tert-butyl (3-amino-1,1-dimethyl-propyl)-carbamate and 5.82 g (42.1 mmol) potassium carbonate are stirred in 60 mL dimethylformamide for 5 days at ambient temperature and 48 hours at 60° C. The reaction mixture is concentrated by evaporation in the rotary evaporator, combined with water and then extracted with ethyl acetate. The combined organic phases are washed successively with water and saturated aqueous sodium chloride solution, dried on sodium sulphate and concentrated by evaporation in the rotary evaporator. 12.7 g (34.6 mmol, quantitative %) 4-(3-tert-butoxycarbonylamino-3-methyl-butylamino)-3-nitro-benzoic acid are obtained.

R_{f}=0.43 [silica gel, petroleum ether/ethyl acetate (1/1)]

MS [ESI (M+H)⁺]=368

### Step 4: 3-amino-4-(3-tert-butoxycarbonylamino-3-methyl-butylamino)-benzoic acid

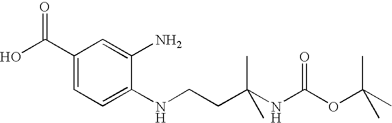

12.7 g (34.6 mmol) 4-(3-tert-butoxycarbonylamino-3-methyl-butylamino)-3-nitro-benzoic acid, 0.850 g Raney nickel in 100 mL methanol are shaken at ambient temperature in an autoclave at 3 bar hydrogen atmosphere for 24 hours. The reaction mixture is filtered and the filtrate is concentrated by evaporation in the rotary evaporator. 10.2 g (30.2 mmol, 87%) 3-amino-4-(3-tert-butoxycarbonylamino-3-methyl-butylamino)-benzoic acid are obtained.

R_{f}=0.22 [silica gel, petroleum ether/ethyl acetate (1/1)]

MS [ESI (M+H)⁺]=338

### Step 5 methyl 1-(3-tert-butoxycarbonylamino-3-methyl-butyl)-2-oxo-2,3-dihydro-1H-benzimidazole-5-carboxylate

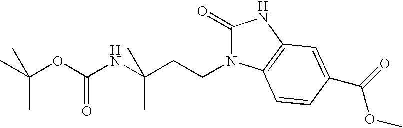

9.80 g (60.5 mmol) 1.1′-carbonyldiimidazole are added at ambient temperature to 10.2 g (30.3 mmol) 3-amino-4-(3-tert-butoxycarbonylamino-3-methyl-butylamino)-benzoic acid in 50 mL tetrahydrofuran. The reaction mixture is stirred for 18 hours at ambient temperature, then combined with methanol and concentrated by evaporation in the rotary evaporator. The residue is dissolved in ethyl acetate, washed successively with aqueous potassium hydrogen sulphate solution (0.5 M) and water, dried on sodium sulphate and concentrated by evaporation in the rotary evaporator. The residue is purified by reversed-phase flash column chromatography {Varian Microsorb C18-reverse-phase [acetonitrile (0.1% trifluoroacetic acid)/water (0.13% trifluoroacetic acid)=20:80→80:20]}, thus obtaining 1.70 g (4.68 mmol, 15%) methyl 1-(3-tert-butoxycarbonylamino-3-methyl-butyl )-2-oxo-2,3-dihydro-1H-benzimidazole-5-carboxylate.

MS [ESI (M+H)⁺]=378

### Step 6 methyl 1-(3-amino-3-methyl-butyl)-2-oxo-2,3-dihydro-1H-benzimidazole-5-carboxylate

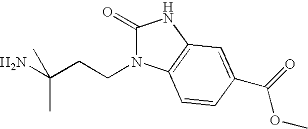

1.15 g (3.05 mmol) methyl 1-(3-tert-butoxycarbonylamino-3-methyl-butyl)-2-oxo-2,3-dihydro-1H-benzimidazole-5-carboxylate are dissolved in 8 mL dichloromethane and combined with 8 mL trifluoroacetic acid. The reaction mixture is stirred for 2 hours at 40° C. and then concentrated by evaporation in the rotary evaporator. The residue is purified by reversed-phase flash column chromatography {Varian Microsorb C18-reverse-phase [acetonitrile (0.1% trifluoroacetic acid)/water (0.13% trifluoroacetic acid)=10:90→100:0]}, thus obtaining 0.800 g (2.90 mmol, 95%) methyl 1-(3-amino-3-methyl-butyl)-2-oxo-2,3-dihydro-1H-benzimidazole-5-carboxylate.

R_{f}=0.36 [silica gel, dichloromethane/methanol/ammonia (90/10/0.1)]

MS [ESI (M+H)⁺]=278

### Step 7: methyl 1-{3-[2-(3-benzenesulphonylamino-phenyl)-2-hydroxy-ethylamino]-3-methyl-butyl}-2-oxo-2,3-dihydro-1H-benzimidazole-5-carboxylate

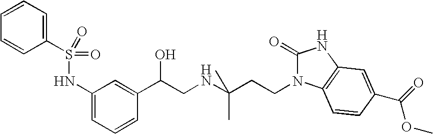

0.300 g (0.895 mmol) N-[3-(2-ethoxy-2-hydroxy-acetyl)-phenyl]-benzenesulphonamide and 0.248 g (0.895 mmol) methyl 1-(3-amino-3-methyl-butyl)-2-oxo-2,3-dihydro-1H-benzimidazole-5-carboxylate are dissolved in 10 mL ethanol and the pH value of the reaction mixture is adjusted to 8-9 with triethylamine. The reaction mixture is refluxed for 16 hours, then cooled to ambient temperature and combined with 0.135 g (0.358 mmol) sodium borohydride. The mixture is stirred for a further 2 hours at ambient temperature and then filtered. The solid obtained is washed with diethyl ether and dried. 0.170 g (0.308 mmol, 34%) methyl 1-{3-[2-(3-benzenesulphonylamino-phenyl)-2-hydroxy-ethylamino]-3-methyl-buty1,1-2-oxo-2,3-dihydro-1H-benzimidazole-5-carboxylate are obtained.

R_{f}=0.47 [silica gel, dichloromethane/methanol/ammonia (90/10/0.1)]

MS [ESI (M+H)⁺]=553

### EXAMPLE 14

### 1-{3-[2-(3-benzenesulphonylamino-phenyl)-2-hydroxy-ethylamino]-3-methyl-butyl}-2-oxo-2,3-dihydro-1H-benzimidazole-5-carboxylic acid-hydrotrifluoroacetate

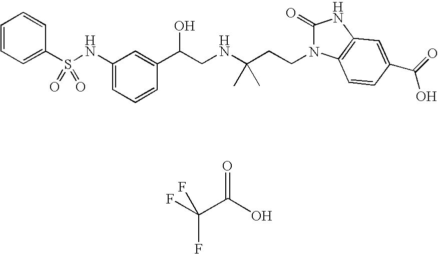

0.100 g (0.181 mmol) methyl 1-{3-[2-(3-benzenesulphonylamino-phenyl)-2-hydroxy-ethylamino]-3-methyl-butyl}-2-oxo-2,3-dihydro-1H-benzimidazole-5-carboxylate are dissolved in 5 mL methanol and combined with 5 mL lithium hydroxide solution (2M in water). The reaction mixture is stirred for 3 hours at ambient temperature and then adjusted to a pH <2 with trifluoroacetic acid. Purification by reversed-phase flash column chromatography {Varian Microsorb C18-reverse-phase [acetonitrile (0.1% trifluoroacetic acid)/water (0.13% trifluoroacetic acid)=10:90→100:0]} yielded 0.090 g (0.138 mmol, 76%) 1-{3-[2-(3-benzenesulphonylamino-phenyl)-2-hydroxy-ethylamino]-3-methyl-butyl}-2-oxo-2,3-dihydro-1H-benzimidazole-5-carboxylic acid trifluoroacetate.

R_{f}=0.24 [silica gel, dichloromethane/methanol/ammonia (80/20/0.1)]

MS [ESI (M+H)⁺]=539

### EXAMPLE15

### N-(63-{2-[1,1-dimethyl-3-(3-methyl-2-oxo-2,3-dihydro-benzimidazol-1-yl)-propylamino]-1-hydroxy-ethyl}-phenyl)-benzenesulphonamide

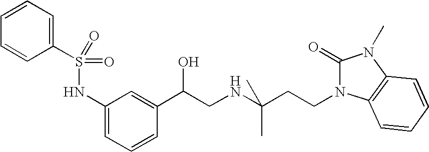

1.00 g (0.2.98 mmol) N-[3-(2-ethoxy-2-hydroxy-acetyl)-phenyl]-benzenesulphonamide and 0.804 g (0.2.98 mmol) 1-(3-amino-3-methyl-butyl)-3-methyl-1,3-dihydro-benzimidazole-2-one hydrochloride are dissolved in 10 mL ethanol and the pH of the reaction mixture is adjusted to 8-9 with triethylamine. The reaction mixture is refluxed for 16 hours, then cooled to ambient temperature and combined with 0.450 g (11.9 mmol) sodium borohydride. The mixture is stirred for a further 2 hours at ambient temperature and then filtered. The solid obtained is washed with diethyl ether, dried and triturated with methanol. 0.620 g (1.22 mmol, 41%) N-(3-{2-[1,1-dimethyl-3-(3-methyl-2-oxo-2,3-dihydro-benzimidazol-1-yl)-propylamino]-1-hydroxy-ethyl}-phenyl)-benzenesulphonamide are obtained.

R_{f}=0.56 [dichloromethane/methanol/ammonia (90/10/0.1)]

MS [ESI (M+H)⁺]=509

### EXAMPLE 16

### Ethyl 1-{3-[2-(3-benzenesulphonylamino-phenyl)-2-hydroxy-ethylamino]-3-methyl-butyl}-1H-benzimidazole4-carboxylate hydrochloride

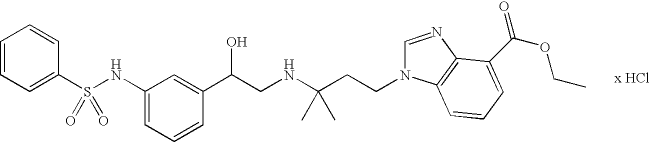

32 mg (0.055 mmol) 1-{3-[2-(3-benzenesulphonylamino-phenyl)-2-hydroxy-ethylamino]-3-methyl-butyl}-1H-benzomidazol-4-carboxylic acid-hydrotrifluoroacetate (Example 5) are dissolved in 10 ml of ethanol and combined with 5 ml of ethanolic HCl (approx. 14 M). The mixture is refluxed for 18 hours and then the solvent is eliminated in vacuo.

Yield: 32 mg (87% of theory)

R_{f}=0.36 [silica gel, dichloromethane/methanol/ammonia (90/10/0.1)]

MS [ESI (M+H)⁺]=551

### EXAMPLE 17

### (2-morpholino-ethyl)(R)-1-{3-[2-(3-benzenesulphonylamino-phenyl)-2-hydroxy-ethylamino]-3-methyl-butyl}-1H-benzimidazole-5-carboxylate dihydrotrifluoroacetate

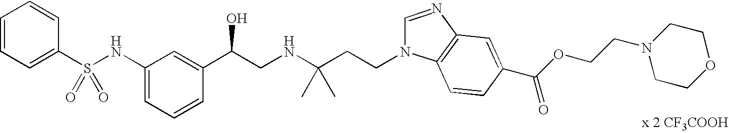

200 mg (0.314 mmol) (R)-1-{3-[2-(3-benzenesulphonylamino-phenyl)-2-hydroxy-ethylamino]-3-methyl-butyl}-1H-benzimidazole-5-carboxylic acid-hydrotrifluoroacetate (Example 7) are dissolved in 3 ml DMF and combined with 126 mg (1.26 mmol) potassium carbonate. The mixture is stirred for 20 minutes at ambient temperature and then 146 mg (0.785 mmol) N-(2-chloroethyl)-morpholine-hydrochlorideare added. The mixture is stirred for another 20 hours at 50° C. and a further 126 mg (1.26 mmol) potassium carbonate and 146 mg (0.785 mmol) N-(2-chloroethyl)-morpholine-hydrochloride are added. After another 3 hours' stirring at 60° C. a little ice water is added and the mixture is carefully acidified with trifluoroacetic acid. The reaction solution is chromatographed on a reversed-phase flash column (Varian Microsorb C18) [acetonitrile (0.1% trifluoroacetic acid)/water (0.13% trifluoroacetic acid)=10:90→100:0].

Yield: 100 mg (37% of theory)

MS [ESI (M+H)⁺]=636

HPLC retention time: 1.73 minutes

### EXAMPLE 18

### (R)-1-{3-[2-(3-benzenesulphonylamino-phenyl)-2-hydroxy-ethylamino]-3-methyl-butyl}-1H-benzimidazole-5-carboxylic acid hydrazide-hydrotrifluoroacetate

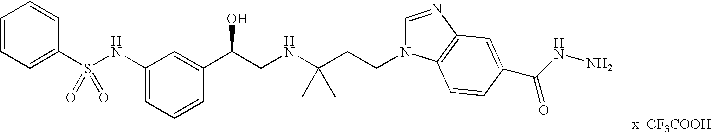

166 mg (0.216 mmol) (R)-1-{3-[2-(3-benzenesulphonylamino-phenyl)-2-hydroxy-ethylamino]-3-methyl-butyl}-1H-benzimidazole-5-carboxylic acid-hydrotrifluoroacetate (Example 7) are dissolved in 3 ml DMF and combined successively with 125 mg (0.653 mmol) N-(3-dimethylaminopropyl)-N′-ethylcarbodiimide-hydrochloride and 81 mg (0.6 mmol) 1-hydroxy-1H-benzotriazole-hydrate. After 60 minutes' stirring at ambient temperature 111 μl (0.653 mmol) DIPEA are added. After another 10 minutes' stirring 5.5 ml (5.5 mmol) of a 1 M hydrazine solution in tetrahydrofuran are added. After 66 hours stirring at ambient temperature the solvent is eliminated in vacuo. The residue is combined with a little ice water and DMF and acidified with trifluoroacetic acid. The solution is chromatographed on a reversed-phase flash column (Varian Microsorb C18) [acetonitrile (0.1% trifluoroacetic acid)/water (0.13% trifluoroacetic acid)=10:90→100:0].

Yield: 70 mg (41% of theory)

MS [ESI (M+H)⁺]=537

HPLC retention time: 1.72 minutes

### EXAMPLE 19

### 1-{3-[(R)-2-(3-benzenesulphonylamino-phenyl)-2-hydroxy-ethylamino]-3-methyl-butyl}-6-methoxy-1H-benzimidazole-5-carboxylic acid-hydrotrifluoroacetate

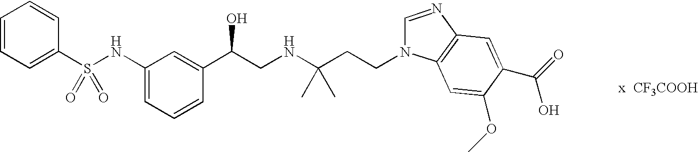

### Step 1: 4 methyl -[acetyl-(3-tert-butoxycarbonylamino-3-methyl-butyl)-amino]-2-methoxy-5-nitro-benzoate

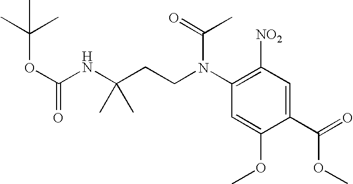

2.68 g (10 mmol) methyl 4-acetylamino-2-methoxy-5-nitrobenzoate are suspended in 15 ml DMF and mixed batchwise with 1.23 g (11 mmol) potassium-tert-butoxide at ambient temperature. 2.92 g (11 mmol) tert. butyl 4,4-dimethyl-2,2-dioxo-[1,2,3]oxathiazinan-3-carboxylate are then added batchwise to the red reaction solution while cooling with ice. The reaction solution is stirred for 21 hours at ambient temperature. Then 250 ml of water are added. 5 ml of 1N hydrochloric acid and 100 ml of ethyl acetate are added. The reaction solution is extracted with ethyl acetate, the organic phase is freed from the solvent in vacuo and the residue is chromatographed on silica gel (petroleum ether/ethyl acetate=2:3).

Yield: 2.65 g (53% of theory)

MS [ESI (M+H)⁺]=454

R_{f}=0.15 [silica gel, petroleum ether/ethyl acetate (1/1)]

HPLC retention time: 3.70 minutes

### Step 2: methyl 4-(3-tert-butoxycarbonylamino-3-methyl-butylamino)-2-methoxy-5-nitro-benzoate

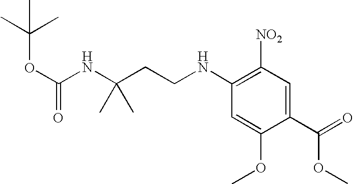

4.7 g (10.3 mmol) methyl 4-[acetyl-(3-tert-butoxycarbonylamino-3-methyl-butyl)-amino]-2-methoxy-5-nitro-benzoate are dissolved in 50 ml of methanol and at ambient temperature 2.2 g (10.4 mmol) 25% sodium methoxide solution in methanol are added dropwise. After 66 hours stirring at ambient temperature the mixture is poured onto 800 ml ice water, neutralised with 1 N hydrochloric acid and extracted with ethyl acetate. The organic phases are combined, washed with saturated saline solution, dried on magnesium sulphate and evaporated to dryness.

Yield: 3.8 g (89% of theory)

MS [ESI (M+H)⁺]=412

R_{f}=0.62 [silica gel, petroleum ether/ethyl acetate (1/1)]

HPLC retention time: 4.27 minutes

### Step 3: methyl 1-(3-amino-3-methyl-butyl)-6-methoxy-1H-benzimidazole-5-carboxylate

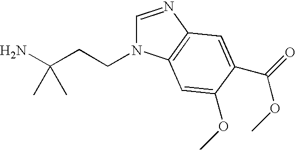

3.8 g (9.24 mmol) methyl 4-(3-tert-butoxycarbonylamino-3-methyl-butylamino)-2-methoxy-5-nitro-benzoate are dissolved in 50 ml formic acid and combined with 3.1 g 500 mg palladium (10% on charcoal). After 16 stirring at ambient temperature 3 g (46 mmol) zinc powder are added. The mixture is stirred for 30 minutes at 60° C. and for 1 hour at 90° C., then for another 16 hours at ambient temperature. After filtration through Celite the filtrate is combined with ethyl acetate and semisaturated sodium hydrogen carbonate solution and extracted with ethyl acetate. The combined organic phases are washed with saturated saline solution and dried on magnesium sulphate. The residue obtained after elimination of the solvent in vacuo is chromatographed on silica gel [dichloromethane/methanol/ammonia (90/9/1)].

Yield: 2.4 g (89% of theory)

MS [ESI (M+H)⁺]=292

R_{f}=0.3 [silica gel, dichloromethane/methanol/ammonia (90/9/1)]

### Step 4: methyl 1-{3-[(R)-2-(3-dibenzolsulphonylamino-phenyl)-2-hydroxy-ethylamino]-3-methyl-butyl}-6-methoxy-1H-benzimidazole-5-carboxylate

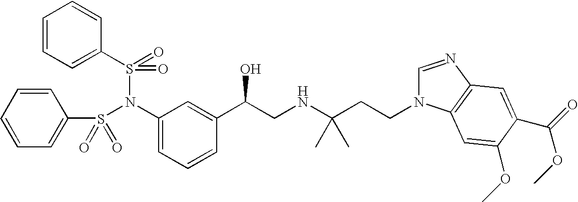

3.2 g (7.83 mmol) N-[(R)-3-oxiranyl-phenyl]-dibenzenesulphonamide (Component 5) are added to 2.4 g (8.24 mmol) methyl 1-(3-amino-3-methyl-butyl)-6-methoxy-1H-benzimidazole-5-carboxylate and heated to 120° C. for 2.5 hours. After cooling to ambient temperature the melt is dissolved in a little dichloromethane and chromatographed on silica gel [dichloromethane/methanol (10/0→9/1)].

Yield: 1.65 g (30% of theory)

MS [ESI (M+H)⁺]=707

HPLC retention time: 2.83 minutes

### Step 5: 1-{3-[(R)-2-(3-benzenesulphonylamino-phenyl)-2-hydroxy-ethylamino]-3-methyl-butyl}-6-methoxy-1H-benzimidazole-5-carboxylic acid-hydrotrifluoroacetate

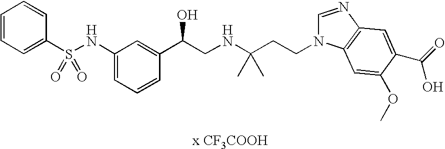

1.65 g (2.33 mmol) methyl 1-{3-[(R)-2-(3-dibenzolsulphonylamino-phenyl)-2-hydroxy-ethylamino]-3-methyl-butyl}-6-methoxy-1H-benzimidazole-5-carboxylate are dissolved in 20 ml of methanol and combined with 4 ml 2 N sodium hydroxide solution. After the addition of 5 ml of tetrahydrofuran the mixture is stirred for 18 hours at ambient temperature. Then the solvent is eliminated in vacuo and the residue is chromatographed on a reversed-phase flash column (Varian Microsorb C18) [acetonitrile (0.1% trifluoroacetic acid)/water (0.13% trifluoroacetic acid)=10:90→100:0].

Yield: 1.1 g (70% of theory)

MS [ESI (M+H)⁺]=553

HPLC retention time: 1.94 minutes

### EXAMPLE 20

### 1-{3-[(R)-2-(3-benzenesulphonylamino-phenyl)-2-hydroxy-ethylamino]-3-methyl-butyl}-6-chloro-1H-benzimidazole-5-carboxylic acid-hydrotrifluoroacetate

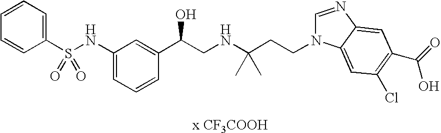

### Step 1: 6-chloro-1H-benzimidazole-5-carboxylic acid

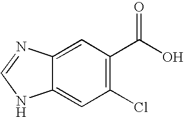

500 mg Raney nickel are added to a solution of 5.0 g (20.3 mmol) 2-chloro-4,5-dinitrobenzoic acid in 50 ml formic acid. The mixture is hydrogenated for 30 hours at ambient temperature and at a hydrogen pressure of 3 bar. Then the catalyst is eliminated by suction filtering and the residue is heated to 100° C. for 10 hours. The solvent is eliminated in vacuo and the residue is triturated with ethyl acetate.

Yield: 3.9 g (98% of theory)

C₈H₅CIN₂O₂ (196.59)

MS [ESI (M+H)⁺]=199, 197

R_{f}=0.03 [silica gel, dichloromethane/methanol/ammonia (80/20/0.1)

### Step 2: ethyl 6-chloro-1H-benzimidazole-5-carboxylate hydrochloride

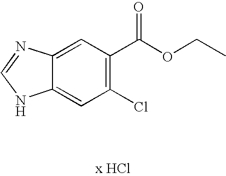

3.9 g (20 mmol) 6-chloro-1H-benzimidazole-5-carboxylic acid are dissolved in 40 ml of ethanol and combined with 15 ml of ethanolic hydrochloric acid. The mixture is refluxed for 72 hours, then the pH is adjusted to basic with ammonia solution and the mixture is applied to silica gel. Filtration through silica gel (dichloromethane/methanol=9:1) yields the product.

Yield: 2.2 g (37% of theory)

C₁₀H₉CIN₂O₃×HCl (261.10)

MS [ESI (M+H)⁺]=225, 223

R_{f}=0.46 [silica gel, dichloromethane/methanol/ammonia (90/10/0.1)

### Step 3: ethyl 1-(3-tert-butoxycarbonylamino-3-methyl-butyl)-6-chloro-1H-benzimidazole-5-carboxylate

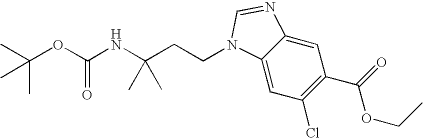

1.21 g (10.8 mmol) potassium-tert-butoxide are added at ambient temperature to 2.20 g (9.79 mmol) ethyl 6-chloro-1H-benzimidazole-5-carboxylate in 12.0 mL N,N-dimethylformamide and the reaction mixture is stirred for 10 minutes. 2.85 g (10.8 mmol) N-tert-butoxycarbonyl-4,4-dimethyl-[1,2,3]oxathiazinane-2,2-dioxide (Hoffmann-La-Roche Patent WO03037327) are added and the mixture is stirred for another 2 hours at ambient temperature. The reaction mixture is combined with water and extracted with ethyl acetate. The combined organic phases are dried on sodium sulphate and freed from the solvent in vacuo. Purification by flash column chromatography (DCM/methanol=100:0→95:5) yields 0.880 g (2.15 mmol, 22% of theory) ethyl 1-(3-tert-butoxycarbonylamino-3-methyl-butyl)-6-chloro-1H-benzimidazole-5-carboxylate.

Yield: 22% of theory

C₂₀H₂₈CIN₃O₄ (409.91)

MS [ESI (M+H)⁺]=412, 410

R_{f}=0.19 [silica gel, petroleum ether/ethyl acetate (3/1)]

In the reaction ethyl 1-(3-tert-butoxycarbonylamino-3-methyl-butyl)-5-chloro-1H-benzimidazole-6-carboxylate is also obtained:

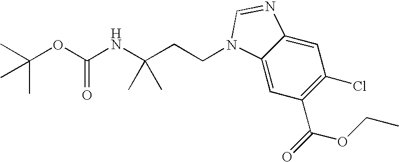

Yield: 25% of theory

C₂₀H₂₈CIN₃O₄ (409.91)

MS [ESI (M+H)⁺]=412, 410

R_{f}=0.23 [silica gel, petroleum ether/ethyl acetate (3/1)]

### Step 4: ethyl 1-(3-amino-3-methyl-butyl)-6-chloro-1H-benzimidazole-5-carboxylate

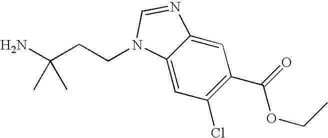

Prepared analogously to Example 5, Step 3 from ethyl 1-(3-tert-butoxycarbonylamino-3-methyl-butyl )-6-chloro-1H -benzimidazole-5-carboxylate in ethanolic hydrochloric acid.

Yield: 90% of theory

C₁₅H₂₀CIN₃O₂ (309.79)

MS [ESI (M+H)⁺]=312, 310

R_{f}=0.16 [silica gel, dichloromethane/methanol/ammonia (90/10/0.1)]

### Step 5: 1-{3-[(R)-2-(3-benzenesulphonylamino-phenyl)-2-hydroxy-ethylamino]-3-methyl-butyl}-6-chloro-1H-benzimidazole-5-carboxylic acid-hydrotrifluoroacetate

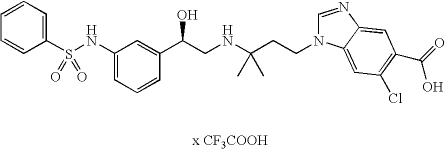

Prepared analogously to Example 6, Step 4 from (R)—N-(3-oxiranyl-phenyl)-benzenesulphonamide and ethyl 3-(3-amino-3-methyl-butyl)-6-chloro-1H-benzimidazole-5-carboxylate in a melt and subsequent saponification with sodium hydroxide solution.

Yield: 29% of theory

C₂₇H₂₉CIN₄O₅S×C₂HF₃O₂ (671.09)

MS [ESI (M+H)⁺]=559, 557

retention time HPLC-MS: 2.13 minutes

### EXAMPLE 21

### 1-{3-[(R)-2-(3-benzenesulphonylamino-phenyl)-2-hydroxy-ethylamino]-3-methyl-butyl}-5-chloro-1H-benzimidazole-6-carboxylic acid-hydrotrifluoroacetate

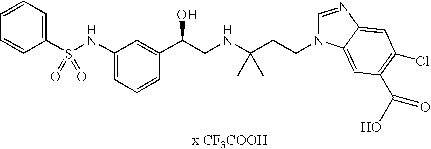

Prepared analogously to Example 20 by reacting ethyl 1-(3-tert-butoxycarbonylamino-3-methyl-butyl )-5-chloro-1H-benzimidazole-6-carboxylate with ethanolic hydrochloric acid, subsequently melting with (R)—N-(3-oxiranyl-phenyl)-benzenesulphonamide and saponification with sodium hydroxide solution.

Yield: 28% of theory

C₂₇H₂₉CIN₄O₅S×C₂HF₃O₃ (671.09)

MS [ESI (M+H)⁺]=559, 557

retention time HPLC-MS: 2.13 minutes

### EXAMPLE 22

### 1-{3-[(R)-2-(3-benzenesulphonylamino-phenyl)-2-hydroxy-ethylamino]-3-methyl-butyl}-4-chloro-1 H-benzimidazole-5-carboxylic acid-hydrotrifluoroacetate

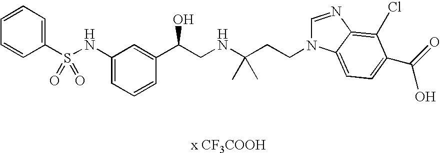

### Step 1: 4-amino-5-methyl-benzimidazole

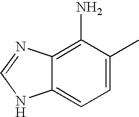

8.0 g (36 mmol) 5-methyl-4-nitro-benzimidazole are dissolved in 50 ml of ethyl acetate and 50 ml of methanol and combined with 500 mg Raney nickel. The mixture is hydrogenated for 18 hours at 3 bar hydrogen pressure. Then the catalyst is removed by suction filtering and the solvent is eliminated in vacuo.

Yield: 5.2 g (98% of theory)

C₈H₉N₃ (147.18)

MS [ESI (M+H)⁺]=148

R_{f}=0.31 [silica gel, dichloromethane/methanol/ammonia (90/9/1)]

### Step 2: 4-chloro-5-methyl-benzimidazole

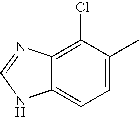

2.68 g (38.9 mmol) sodium nitrite dissolved in 10 ml of water are added dropwise at 5° C. to a solution of 5.2 g (35.3 mmol) 4-amino-5-methyl-benzimidazole in 170 ml semiconc. hydrochloric acid. After 5 minutes stirring 34.9 g (353 mmol) copper(I)-chloride dissolved in 85 ml conc. hydrochloric acid are added dropwise. The mixture is slowly heated to ambient temperature and then heated to 60° C. for 30 minutes. Then conc. ammonia solution is added until a basic pH is achieved. Ethyl acetate is added, the insoluble matter is filtered off and the filtrate is extracted with ethyl acetate. The combined organic phases are dried on sodium sulphate and evaporated to dryness. The residue is chromatographed on silica gel [dichloromethane/methanol/ammonia (100/0/0→90/10/0.1)].

Yield: 400 mg (7% of theory)

C₈H₇CIN₂ (166.61)

MS [ESI (M+H)⁺]=167, 165

R_{f}=0.24 [silica gel, dichloromethane/methanol/ammonia (95/5/0.1)]

Step 3: ethyl 4-chloro-benzimidazole-5-carboxylate hydrochloride

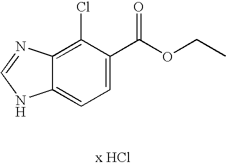

350 mg (2.1 mmol) 4-chloro-5-methyl-benzimidazole are suspended in 10 ml tert-butanol and 10 ml of water and combined with 0.9 g (5.7 mmol) potassium permanganate. The reaction mixture is stirred for 4 hours at 75° C. Another 0.9 g (5.7 mmol) potassium permanganate are added. The mixture is stirred for another 4 hours at 75° C., then cooled to ambient temperature and 1 g sodium sulphite is added. The reaction mixture is filtered through silica gel and the filtrate is freed from the solvent in vacuo. The residue is dissolved in 10 ml of ethanol and combined with 3 ml of ethanolic hydrochloric acid. The mixture is refluxed for 4 hours. The precipitate is suction filtered and washed with ether.

Yield: 480 mg (88% of theory)

C₁₀H₉CIN₂O₂×HCl (261.10)

MS [ESI (M+H)⁺]=227, 225

R_{f}=0.22 [silica gel, dichloromethane/methanol/ammonia (95/5/0.1)]

### Step 4: ethyl 1-(3-tert-butoxycarbonylamino-3-methyl-butyl)-4-chloro-1H-benzimidazole-5-carboxylate

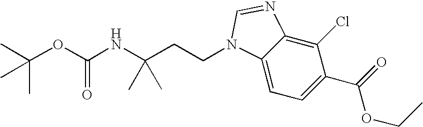

Prepared analogously to Example 20, Step 3 from ethyl 4-chloro-benzimidazole-5-carboxylate and tert. butyl 4,4-dimethyl-2,2-dioxo-[1,2,3]oxathiazinane-3-carboxylate.

Yield: 33% of theory

C₂₀H₂₈CIN₃O₄ (409.91)

MS [ESI (M+H)⁺]=412, 410

R_{f}=0.2 [silica gel, petroleum ether/ethyl acetate (1/1)]

In the reaction ethyl 1-(3-tert-butoxycarbonylamino-3-methyl-butyl)-7-chloro-1H-benzimidazole-6-carboxylate is also obtained:

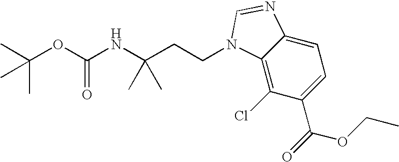

Yield: 24% of theory

C₂₀H₂₈CIN₃O₄ (409.91)

MS [ESI (M+H)⁺]=412, 410

R_{f}=0.28 [silica gel, petroleum ether/ethyl acetate (1/1)]

### Step 5: 1-{3-[(R)-2-(3-benzenesulphonylamino-phenyl)-2-hydroxy-ethylamino]-3-methyl-butyl}-4-chloro-1H-benzimidazole-5-carboxylic acid-hydrotrifluoroacetate

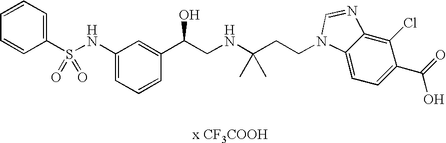

Prepared analogously to Example 20 by reacting ethyl 1-(3-tert-butoxycarbonylamino-3-methyl-butyl )-4-chloro-1H-benzimidazole-5-carboxylate with ethanolic hydrochloric acid, subsequently melting with (R)—N-(3-oxiranyl-phenyl)-benzenesulphonamide and saponification with sodium hydroxide solution.

Yield: 32% of theory

C₂₇H₂₉CIN₄O₅S×C₂HF₃O₂ (671.09)

MS [ESI (M+H)⁺]=559, 557

R_{f}=0.06 [silica gel, dichloromethane/methanol/ammonia (85/15/0.1)]

### EXAMPLE 23

### 1-{3-[(R)-2-(3-benzenesulphonylamino-phenyl)-2-hydroxy-ethylamino]-3-methyl-butyl}-7-chloro-1H-benzimidazole-6-carboxylic acid-hydrotrifluoroacetate

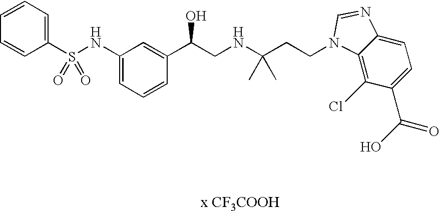

Prepared analogously to Example 20 by reacting ethyl 1-(3-tert-butoxycarbonylamino-3-methyl-butyl )-7-chloro-1H-benzimidazole-6-carboxylate with ethanolic hydrochloric acid, subsequent melting with (R)—N-(3-oxiranyl-phenyl)-benzenesulphonamide and saponification with sodium hydroxide solution.

Yield: 31% of theory

C₂₇H₂₉CIN₄O₅S×C₂HF₃O₃ (671.01)

MS [ESI (M+H)⁺]=559, 557

R_{f}=0.17 [silica gel, dichloromethane/methanol/ammonia (85/15/0.1)]

### EXAMPLE 24

### 1-{3-[(R)-2-(3-benzenesulphonylamino-phenyl)-2-hydroxy-ethylamino]-3-methyl-butyl}-6-hydroxy-1H-benzimidazole-5-carboxylic acid-hydrotrifluoroacetate

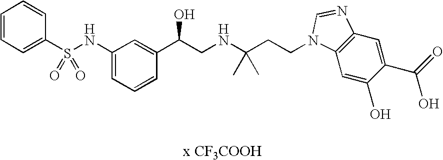

0.400 g (666 mmol) 1-{3-[(R)-2-(3-benzenesulphonylamino-phenyl)-2-hydroxy-ethyl-amino]-3-methyl-butyl}-6-methoxy-1H-benzimidazole-5-carboxylic acid-hydrotrifluoroacetate (Example 19) are dissolved in 1.5 ml N,N-dimethylacetamide and combined with 0.208 g (86.1 mmol) piperazine. The reaction mixture is stirred for 3 hours at 150° C., acidified with trifluoroacetic acid while cooling with ice and the precipitate formed is dissolved by the addition of acetonitrile and N,N-dimethylformamide. Purification by reversed-phase flash column chromatography (Varian Microsorb C18) [acetonitrile (0.1% trifluoroacetic acid)/water (0.13% trifluoroacetic acid)=10:90→100:0] yielded 0.653 g(392 mmol, 61% of theory) 1-{3-[(R)-2-(3-benzenesulphonylamino-phenyl)-2-hydroxy-ethylamino]-3-methyl-butyl}-6-hydroxy-1H-benzimidazole-5-carboxyl ic acid-hydrotrifluoroacetate.

MS [ESI (M+H)⁺]=539

HPLC retention time: 1.95 minutes

What is claimed is:

1. A compound of the formula 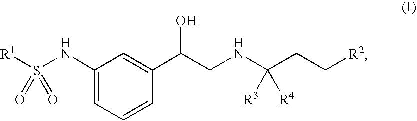
wherein
R¹ denotes a C₁₋₄-alkyl, di-(C₁₃-alkyl)-amino, thienyl, pyridyl or phenyl group,
wherein the phenyl group may be substituted by one to three fluorine, chlorine or bromine atoms or one to three C₁₋₃-alkyl, C₁₋₃-alkyloxy, trifluoromethoxy or difluoromethoxy groups, wherein the substituents may be identical or different
R² denotes a benzimidazolyl or 1,3-dihydrobenzimidazol-2-one group,
each of which may be substituted by one or two fluorine, chlorine or bromine atoms or one or two C₁₋₃-alkyl, hydroxy, methoxy, trifluoromethoxy, difluoromethoxy, carboxy, C₁₋₄-alkyloxy-carbonyl, ω-morpholin-4-yl-C₂₋₄-alkyloxy-carbonyl, hydrazinocarbonyl or amino groups, wherein the substituents may be identical or different or
wherein two adjacent carbon atoms may be bridged by a —CH═CH—CH═CH— group,
and R³ and R⁴, which may be identical or different, each denote a C₁₋₃-alkyl group,
wherein the alkyl groups contained in the above-mentioned groups may be straight-chain or branched,
and excluding the compounds
N-(3-{2-[3-(5-amino-benzimidazol-1-yl)-1,1-dimethyl-propylamino]-1-hydroxy-ethyl}-phenyl)-benzenesulphonamide,
1-{3-[2-(3-benzenesulphonylamino-phenyl)-2-hydroxy-ethylamino]-3-methyl-butyl}-1H-benzimidazole-5-carboxylic acid and
ethyl 1-{3-[2-(3-benzenesuphonylamino-phenyl)-2-hydroxy-ethylamino]-3-methyl-butyl}-1H-benzimidazole-5-carboxylate;
or a salt thereof.

2. A compound of the formula (I) according to claim 1, wherein
R², R³ and R⁴ are defined as in claim 1 and
R¹ denotes a phenyl group, which may be substituted by a fluorine, chlorine or bromine atom or a C₁₋₃-alkyl, C₁₋₃-alkyloxy, trifluoromethoxy or difluoromethoxy group,
with the exclusion of the compounds
N-(3-{2-[3-(5-amino-benzimidazol-1-yl)-1,1-dimethyl-propylamino]-1-hydroxy-ethyl}-phenyl)-benzenesulphonamide,
1-{3-[2-(3-benzenesulphonylamino-phenyl)-2-hydroxy-ethylamino]-3-methyl-butyl}-1H-benzimidazole-5-carboxylic acid and
ethyl 1-{3-[2-(3-benzenesuphonylamino-phenyl)-2-hydroxy-ethylamino]-3-methyl-butyl}-1H-benzimidazole-5-carboxylate;
or a salt thereof.

3. A compound of the formula (I) according to claim 2, wherein
R² is defined as in claim 1,
R¹ denotes a phenyl group
and R³ and R⁴ each represent a methyl group,
with the exclusion of the compounds
N-(3-{2-[3-(5-amino-benzimidazol-1-yl)-1,1-dimethyl-propylamino]-1-hydroxy-ethyl}-phenyl)-benzenesulphonamide,
1-{3-[2-(3-benzenesulphonylamino-phenyl)-2-hydroxy-ethylamino]-3-methyl-butyl}-1H-benzimidazole-5-carboxylic acid and
ethyl 1-{3-[2-(3-benzenesuphonylamino-phenyl)-2-hydroxy-ethylamino]-3-methyl-butyl}-1H-benzimidazole-5-carboxylate;
or a salt thereof.

4. A compound of the formula (I) according to claim 1, wherein
R¹ denotes a phenyl group,
R² denotes a benzimidazol-1-yl or 1,3-dihydrobenzimidazol-2-on-1-yl group,
each of which may be substituted by one or two fluorine, chlorine or bromine atoms or a C₁₋₃-alkyl, hydroxy, methoxy, trifluoromethoxy, difluoromethoxy, carboxy, C₁₋₄-alkyloxy-carbonyl, ω-morpholin-4-yl-C₂₋₄-alkyloxy-carbonyl, hydrazinocarbonyl or amino group or
wherein two adjacent carbon atoms may be bridged by a —CH═CH—CH═CH— group,
and R³ and R⁴ each represent a methyl group,
wherein the alkyl groups contained in the above-mentioned groups may be straight-chain or branched,
and with the exclusion of the compounds
N-(3-{2-[3-(5-amino-benzimidazol-1-yl)-1,1-dimethyl-propylamino]-1-hydroxy-ethyl}-phenyl)-benzenesulphonamide, 1-{3-[2-(3-benzenesulphonylamino-phenyl)-2-hydroxy-ethylamino]-3-methyl-butyl}-1H-benzimidazole-5-carboxylic acid and
ethyl 1-{3-[2-(3-benzenesuphonylamino-phenyl)-2-hydroxy-ethylamino]-3-methyl- butyl}-1H-benzimidazole-5-carboxylate;
or a salt thereof.

5. A compound according to claim 1, wherein
R² denotes a benzimidazol-1-yl group,
which may be substituted by one or two fluorine, chlorine or bromine atoms or a C₁₋₃-alkyl, hydroxy, methoxy, trifluoromethoxy, difluoromethoxy, carboxy, C₁₋₄-alkyloxy-carbonyl, ω-morpholin-4-yl-C₂₋₄-alkyloxy-carbonyl, hydrazino-carbonyl or amino group, wherein the substituents may be identical or different or
wherein two adjacent carbon atoms may be bridged by a —CH═CH—CH═CH— group,
wherein the alkyl groups contained in the above-mentioned groups may be straight-chain or branched,
and with the exclusion of the compounds
N-(3-{2-[3-(5-amino-benzimidazol-1-yl)-1,1-dimethyl-propylamino]-1-hydroxy-ethyl}-phenyl)-benzenesulphonamide,
1-{3-[2-(3-benzenesulphonylamino-phenyl)-2-hydroxy-ethylamino]-3-methyl-butyl}-1H-benzimidazole-5-carboxylic acid and
ethyl 1-{3-[2-(3-benzenesuphonylamino-phenyl)-2-hydroxy-ethylamino]-3-methyl-butyl}-1H-benzimidazole-5-carboxylate;
or a salt thereof.

6. A compound according to claim 1, wherein
R¹ denotes a phenyl group,
R² denotes a benzimidazol-1-yl group,
each of which may be substituted by one or two fluorine, chlorine or bromine atoms or one or two C₁₋₃-alkyl, hydroxy, methoxy, carboxy, C₁₋₄-alkyloxy-carbonyl, ω-morpholin-4-yl-C₂₋₄-alkyloxy-carbonyl, or hydrazinocarbonyl groups, wherein the substituents may be identical or different or
wherein two adjacent carbon atoms may be bridged by a —CH═CH—CH═CH— group,
and R³ and R⁴ each represent a methyl group,
wherein the alkyl groups contained in the above-mentioned groups may be straight-chain or branched,
with the exclusion of the compounds
1-{3-[2-(3-benzenesulphonylamino-phenyl)-2-hydroxy-ethylamino]-3-methyl-butyl}-1H-benzimidazole-5-carboxylic acid and
ethyl 1-{3-[2-(3-benzenesulphonylamino-phenyl)-2-hydroxy-ethylamino]-3-methyl-butyl}-1H-benzimidazole-5-carboxylate;
or a salt thereof.

7. A compound according to one of claims 1 to 6, characterised in that it is the (R)-enantiomer of formula

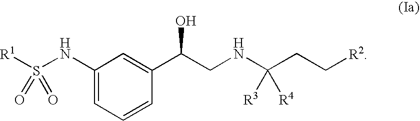

8. A compound according to one of claims 1 to 6, characterised in that it is the (S)-enantiomer of formula

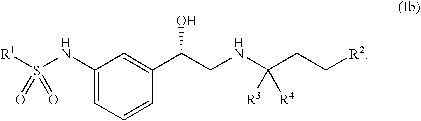

9. A compound selected from the group consisting of:
N-(3-{2-[3-(6-amino-benzimidazol-1-yl)-1,1-dimethyl-propylamino]-1-hydroxy-ethyl}-phenyl)-benzenesulphonamide,
ethyl 1-{3-[2-(3-benzenesulphonylamino-phenyl)-2-hydroxy-ethylamino]-3-methyl-butyl}-1H-benzimidazole-6-carboxylate,
1-{3-[2-(3-benzenesulphonylamino-phenyl)-2-hydroxy-ethylamino]-3-methyl-butyl}-1H-benzimidazole-6-carboxylic acid,
(2-morpholino-ethyl)1-{3-[2-(3-benzenesulphonylamino-phenyl)-2-hydroxy-ethylamino]-3-methyl-butyl}-1H-benzimidazole-5-carboxylate,
1-{3-[2-(3-benzenesulphonylamino-phenyl)-2-hydroxy-ethylamino]-3-methyl-butyl}-1H-benzimidazole-5-carboxylic acid hydrazide,
1-{3-[2-(3-benzenesulphonylamino-phenyl)-2-hydroxy-ethylamino]-3-methyl-butyl}-6-methoxy-1H-benzimidazole-5-carboxylic acid,
1-{3-[2-(3-benzenesulphonylamino-phenyl)-2-hydroxy-ethylamino]-3-methyl-butyl}-6-chloro-1H-benzimidazole-5-carboxylic acid,
1-{3-[2-(3-benzenesulphonylamino-phenyl)-2-hydroxy-ethylamino]-3-methyl-butyl}-5-5chloro-1H-benzimidazole-6-carboxylic acid,
1-{3-[2-(3-benzenesulphonylamino-phenyl)-2-hydroxy-ethylamino]-3-methyl-butyl}-4-chloro-1H-benzimidazole-5-carboxylic acid,
1-{3-[2-(3-benzenesulphonylamino-phenyl)-2-hydroxy-ethylamino]-3-methyl-butyl}-7-chloro-1H-benzimidazole-6-carboxylic acid and
1-{3-[2-(3-benzenesulphonylamino-phenyl)-2-hydroxy-ethylamino]-3-methyl-butyl}-6-hydroxy-1H-benzimidazole-5-carboxylic acid
or a salt thereof.

10. A compound selected from the group consisting of:
N-(3-{2-[3-(6-amino-benzimidazol-1-yl)-1,1-dimethyl-propylamino]-1-hydroxy-ethyl}-phenyl)-benzenesulphonamide,
ethyl (R)-1-{3-[2-(3-benzenesuphonylamino-phenyl)-2-hydroxy-ethylamino]-3-methyl-butyl}-1H-benzimidazole-6-carboxylate,
(R)-1-{3-[2-(3-benzenesulphonylamino-phenyl)-2-hydroxy-ethylamino]-3-methyl-butyl}-1H-benzimidazole-6-carboxylic acid,
(2-morpholino-ethyl)(R)-1-{3-[2-(3-benzenesulphonylamino-phenyl)-2-hydroxy-ethylamino]-3-methyl-butyl}-1H-benzimidazole-5-carboxylate,
(R)-1-{3-[2-(3-benzenesulphonylamino-phenyl)-2-hydroxy-ethylamino]-3-methyl-butyl}-1H-benzimidazole-5-carboxylic acid hydrazide,
1-{3-[(R)-2-(3-benzenesulphonylamino-phenyl)-2-hydroxy-ethylamino]-3-methyl-butyl}-6-methoxy-1H-benzimidazole-5-carboxylic acid,
1-{3-[(R)-2-(3-benzenesulphonylamino-phenyl)-2-hydroxy-ethylamino]-3-methyl-butyl}-6-chloro-1H-benzimidazole-5-carboxylic acid,
1-{3-[(R)-2-(3-benzenesulphonylamino-phenyl)-2-hydroxy-ethylamino]-3-methyl-butyl}-5-chloro-1H-benzimidazole-6-carboxylic acid,
1-{3-[(R)-2-(3-benzenesulphonylamino-phenyl)-2-hydroxy-ethylamino]-3-methyl-butyl}-4-chloro-1H-benzimidazole-5-carboxylic acid,
1-{3-[(R)-2-(3-benzenesulphonylamino-phenyl)-2-hydroxy-ethylamino]-3-methyl-butyl}-7-chloro-1H-benzimidazole-6-carboxylic acid and
1-{3-[(R)-2-(3-benzenesulphonylamino-phenyl)-2-hydroxy-ethylamino]-3-methyl-butyl}-6-hydroxy-1H-benzimidazole-5-carboxylic acid
or a salt thereof.

11. The compounds
(R)-1-{3-[2-(3-benzenesulphonylamino-phenyl)-2-hydroxy-ethylamino]-3-methyl-butyl}-1H-benzimidazole-5-carboxylate ethyl and
(R)-1-{3-[2-(3-benzenesulphonylamino-phenyl)-2-hydroxy-ethylamino]-3-methyl-butyl}-1H-benzimidazole-5-carboxylic acid
and the salts thereof.

12. A physiologically acceptable salt of a compound according to claims 1 to 11.

13. A pharmaceutical composition comprising a compound according to claim 1, 2, 3, 4, 5, 6, 7, 8, 9, 10 or 11, and a pharmaceutically acceptable carrier.

14. A method for treating a disease which is treatable by stimulation of beta-3-receptors, which method comprises administering to a host in need of such treatment a therapeutically effective amount of a compound according to claim 1, 2, 3, 4, 5, 6, 7, 8, 9, 10 or 11.
